(19) United States
(12) Patent Application Publication
(10) Pub. No.: US 2023/0018937 A1
(43) Pub. Date: Jan. 19, 2023

(54) LEAFLET COMMISSURE ASSEMBLIES AND ASSEMBLY METHODS FOR A PROSTHETIC HEART VALVE

(71) Applicant: Edwards Lifesciences Corporation, Irvine, CA (US)

(72) Inventors: Tamir S. Levi, Zikhron Yaakov (IL); Nikolay Gurovich, hadera (IL)

(21) Appl. No.: 17/936,292

(22) Filed: Sep. 28, 2022

Related U.S. Application Data
(63) Continuation of application No. PCT/US2021/023705, filed on Mar. 23, 2021.
(60) Provisional application No. 63/003,085, filed on Mar. 31, 2020.

(51) Int. Cl. A61F 2/24

(52) U.S. Cl. CPC A61F 2/2418; A61F 2220/0075

(57) ABSTRACT

A prosthetic heart valve including an annular frame and a plurality of leaflets is disclosed. The annular frame includes interconnected struts defining a plurality of rows of cells arranged between an outflow and inflow end of the frame and including an upper row of cells at the outflow end. The frame further includes a plurality of commissure windows formed between axially extending window struts of the frame, each commissure window arranged between axially extending window struts of two adjacent cells and having a commissure-receiving portion that is spaced away from an upper end of the two adjacent cells and an open end. Each leaflet includes opposing commissure tabs, each commissure tab being paired with an adjacent commissure tab of an adjacent leaflet to form a commissure, each commissure arranged within the commissure-receiving portion of a respective commissure window, the open end of the commissure window configured to receive the commissure.

### CROSS REFERENCE TO RELATED APPLICATIONS

This application is a continuation of PCT Patent Application No. PCT/US2021/023705, filed Mar. 23, 2021, which claims the benefit of U.S. Provisional Application No. 63/003,085, filed Mar. 31, 2020, all of which are incorporated herein by reference.

### FIELD

The present disclosure relates to prosthetic heart valves, including frames having a row of elongated cells, and to methods and assemblies for forming commissures with leaflets of such prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery device and advanced through the patient's vasculature (e.g., through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery device so that the prosthetic valve can self-expand to its functional size.

Prosthetic valves that rely on a mechanical actuator for expansion can be referred to as “mechanically expandable” prosthetic heart valves. The actuator typically takes the form of pull cables, sutures, wires and/or shafts that are configured to transmit expansion forces from a handle of the delivery apparatus to the prosthetic valve.

Most expandable, transcatheter heart valves comprise a cylindrical metal frame or stent and prosthetic leaflets mounted inside the frame. The leaflets may be attached to the frame at commissure tabs of the leaflets. For example, a commissure may be formed by connecting the commissure tabs of two adjacent leaflets to one another, and in some embodiments, to a flexible sheet or attachment member configured to couple to a commissure support portion of the frame. The commissure can then be attached to the commissure support portion of the frame via a fastener, such as a suture.

Transcatheter heart valves are conventionally used for mid to high expansion diameters, for example in the range of 23 to 29 mm. Smaller diameter valves, such as 20 mm prosthetic valves, are used less frequently due to a variety of challenges that arise from the smaller sized cells of their frame. For example, the smaller cells of such a frame may interfere with blood flow into the coronary ostia following a valve-in-valve (e.g., prosthetic valve-in-prosthetic-valve) procedure. As another example, the smaller sized cells may inhibit the passage therethrough of optional coronary re-access devices, at a later time, following valve implantation.

To at least partially address these issues, the upper row of cells of the prosthetic valve frame, which are arranged proximate to the outflow end of the frame, can be elongated relative to the other rows of cells of the frame. However, the leaflet assembly of the prosthetic valve, including commissures attached to the frame, may at least partially block the openings of the upper rows of cells, thereby reducing access for blood flow and coronary re-access devices.

Accordingly, a need exists for improved prosthetic heart valves and commissure attachment assemblies that retain the leaflets of the prosthetic valve in a lower position within the frame. This lower position may maintain a relatively open cell area within the upper cells, proximate to the outflow end of the frame, thereby allowing increased blood flow to the coronary ostia, and passage of a coronary re-access device, once the prosthetic valve is implanted.

### SUMMARY

Described herein are embodiments of prosthetic heart valves and methods for assembling prosthetic heart valves including an annular frame and a leaflet assembly. In some embodiments, at least partially pre-assembled commissures, each including a pair of commissure tabs of adjacent leaflets of a leaflet assembly, can be arranged within a commissure window of the annular frame or a commissure support element configured to be coupled to a portion of the annular frame configured to receive the commissure support element. The commissure windows, portions of the annular frame configured to receive the commissure support elements, and/or the commissure support elements can be configured to position the commissure within a lower portion of upper, elongated cells of the annular frame.

In one representative embodiment, a method of assembling a prosthetic heart valve including an annular frame and a leaflet assembly includes: inserting a commissure, pre-assembled outside the annular frame, into a commissure window of the frame via an opening in an upper or lower portion of the commissure window, wherein the commissure comprises a pair of secured together commissure tabs of adjacently arranged leaflets of the leaflet assembly, wherein each commissure window is formed by one or more axially extending struts of two adjacent cells in an upper row of cells of the frame, the upper row of cells arranged at an outflow end of the frame and having an elongated length, in an axial direction relative to a central longitudinal axis of the frame, relative to cells in remaining rows of cells of the frame. The method further includes securing the commissure within a commissure-receiving portion of the commissure window via one or more fasteners such that the commissure is arranged within a lower portion of the upper rows of cells and an open area that is unblocked from leaflets is formed within an upper portion of the upper row of cells, the upper portion arranged closer to the outflow end than the lower portion.

In another representative embodiment, a prosthetic heart valve includes: an annular frame including a plurality of interconnected struts defining a plurality of rows of cells, the plurality of rows arranged between an outflow end and an inflow end of the frame and including an upper row of cells arranged at the outflow end and a lower row of cells arranged at the inflow end. The annular frame further includes a plurality of commissure windows formed between axially extending window struts of the frame, each commissure window arranged between axially extending window struts of two adjacent cells of the upper row of open cells, each commissure window having a commissure-receiving portion that is spaced away from an upper end of the two adjacent cells, the upper end arranged at the outflow end of the frame, and an open end. The prosthetic heart valve further includes a leaflet assembly comprising a plurality of leaflets, each leaflet comprising opposing commissure tabs on opposite sides of the leaflet, each commissure tab being paired with an adjacent commissure tab of an adjacent leaflet to form a commissure, where each commissure is arranged within the commissure-receiving portion of a respective commissure window, and where the open end of the commissure window is configured to receive the commissure therethrough.

In some embodiments, cells of the upper row of cells are elongated, in an axial direction relative to a central longitudinal axis of the frame, relative to cells of remaining rows of cells of the plurality of rows of cells, including the lower row of cells.

In another representative embodiment, a prosthetic heart valve includes: an annular frame including a plurality of interconnected struts defining a plurality of rows of cells, the plurality of rows of cells arranged between an outflow end and an inflow end of the frame and including an upper row of cells arranged at the outflow end that are elongated in an axial direction relative to cells of remaining rows of cells of the plurality of rows of cells, the axial direction relative to a central longitudinal axis of the frame. The prosthetic heart valve further includes a plurality of leaflets situated within the frame, each leaflet comprising opposing commissure tabs on opposite sides of the leaflet, each commissure tab being paired with an adjacent commissure tab of an adjacent leaflet to form a commissure. The struts defining the upper row of cells further define a plurality of open commissure windows that are open at an upper end and formed between adjacent elongated cells of the upper row of cells, each commissure window formed between two axially extending window struts that are spaced apart from one another in a circumferential direction, each commissure window comprising a lower region configured to receive commissure tabs of the commissure and an upper region arranged at the outflow end of the frame.

In another representative embodiment, a prosthetic heart valve includes an annular frame including a plurality of interconnected struts defining a plurality of rows of cells, the plurality of rows arranged between an outflow end and an inflow end of the frame and including an upper row of cells arranged at the outflow end that are elongated in an axial direction relative to cells of remaining rows of cells of the plurality of rows of cells, the axial direction relative to a central longitudinal axis of the frame. The prosthetic heart valve further includes a plurality of leaflets situated within the frame, each leaflet comprising opposing commissure tabs on opposite sides of the leaflet, each commissure tab being paired with an adjacent commissure tab of an adjacent leaflet to form a commissure. Struts defining the upper row of cells further define a plurality of open commissure windows that are open at a lower end and formed between adjacent elongated cells of the upper row of cells, within a lower portion of the adjacent elongated cells, each commissure window configured to receive a commissure therein and formed by two axially extending window struts that are spaced apart from one another in a circumferential direction, the two axially extending window struts connected to an upper axial strut via an upper edge of the commissure window and to lower angled struts of the upper row of cells, the upper axial strut and the two axially extending window struts together forming an axially extending strut defining an axial side of each of two adjacent elongated cells.

In another representative embodiment, a prosthetic heart valve includes an annular frame including a plurality of interconnected struts defining a plurality of rows of cells, the plurality of rows of cells arranged between an outflow end and an inflow end of the frame and including an upper row of cells arranged at the outflow end that are elongated in an axial direction relative to cells of remaining rows of cells of the plurality of rows of cells, the axial direction relative to a central longitudinal axis of the frame. The prosthetic heart valve further includes a plurality of leaflets situated within the frame, each leaflet comprising opposing commissure tabs on opposite sides of the leaflet, each commissure tab being paired with an adjacent commissure tab of an adjacent leaflet to form a commissure. Struts defining the upper row of cells further define a plurality of open commissure windows, each commissure window being open at an upper end and formed between two adjacent elongated cells of the upper row of cells, within a lower portion of the two adjacent elongated cells, each commissure window split into a first open window portion and a second open window portion, each of the first and second open window portions configured to receive a different one of the commissure tabs of the commissure therein, where the first open window portion is formed between a central, axially extending strut and a first axial strut arm connected to and laterally offset, in a circumferential direction relative to a circumference of the frame, from the axially extending strut, and where the second open window portion is formed between the axially extending strut and a second axial strut arm connected to a laterally offset from the axially extending strut.

In another representative embodiment, a prosthetic heart valve includes an annular frame including a plurality of interconnected struts defining a plurality of rows of cells, the plurality of rows of cells arranged between an outflow end and an inflow end of the frame and including an upper row of cells arranged at the outflow end and a lower row of cells arranged at the inflow end, wherein cells of the upper row of cells are elongated, in an axial direction relative to a central longitudinal axis of the frame, relative to cells of remaining rows of cells of the plurality of rows of cells, including the lower row of cells. The annular frame further includes a plurality of commissure windows, each commissure window defined by a closed frame, the closed frame including a wider, first portion and a narrower, second portion, the first portion connected to an upper elongated strut junction of the frame via a bendable strut portion, the upper elongated strut junction being a junction between two angled struts of two adjacent elongated cells of the upper row of cells and an axially extending strut arranged between the two adjacent elongated cells, where the commissure window is configured to bend, via its bendable strut portion, from an extended configuration to a bent configuration where the commissure window overlaps the axially extending strut. The prosthetic heart valve further includes a leaflet assembly comprising a plurality of leaflets, each leaflet comprising opposing commissure tabs on opposite sides of the leaflet, each commissure tab being paired with an adjacent commissure tab of an adjacent leaflet to form a commissure, where each commissure is arranged within a respective second portion of a respective commissure window.

In another representative embodiment, a method for assembling a prosthetic heart valve comprising an annular frame and a leaflet assembly includes bending a commissure window of the annular frame to a first bent orientation between a fully extended and a fully bent configuration, where the commissure window is defined by a closed strut frame including a wider, first portion and a narrower, second portion, the first portion connected to an upper elongated strut junction of the annular frame via a bendable strut portion, the upper elongated strut junction being a junction between two angled struts of two adjacent elongated cells of an upper row of elongated cells arranged at an outflow end of the annular frame and an axially extending strut arranged between the two adjacent elongated cells, where in the fully extended configuration the bendable strut portion is not bent and the commissure window extends in a axial direction, relative to a central longitudinal axis of the annular frame, outward and away from the outflow end of the annular frame, and where in the fully bent configuration the commissure window overlaps the axially extending strut. The method further includes inserting a commissure, at least partially pre-assembled outside the annular frame, into the commissure window, where the commissure comprises a pair of secured together commissure tabs of adjacently arranged leaflets of the leaflet assembly. The method further includes bending the commissure window to the fully bent configuration, so that the commissure window extends in parallel with the axially extending strut and is arranged radially inward of the axially extending strut, and securing the commissure window, in its fully bend configuration, to the annular frame.

In another representative embodiment, a method of assembling a prosthetic heart valve including an annular frame and a leaflet assembly includes inserting a commissure, at least partially pre-assembled outside the annular frame, into a leaflet-receiving window of a commissure support element through an opening in the leaflet-receiving window, where the commissure comprises a pair of secured together commissure tabs of adjacently arranged leaflets of the leaflet assembly, where the leaflet-receiving window is formed by at least a portion of two axially extending members of the commissure support element that are open at a first end and connected at an opposing, second end by a connecting member, and where the commissure support element includes a coupling portion connected to the leaflet-receiving window and adapted to couple to a portion of the annular frame forming two adjacent cells in an upper row of cells of the annular frame, the upper row of cells arranged at an outflow end of the annular frame and having an elongated length, in an axial direction relative to a central longitudinal axis of the annular frame, relative to cells in remaining rows of cells of the annular frame. The method further includes attaching the commissure support element to the portion of the annular frame such that the leaflet-receiving window, and the commissure arranged therein, are arranged within a lower portion of the upper rows of cells and an open area that is unblocked from leaflets is formed within an upper portion of the upper row of cells.

In another representative embodiment, a prosthetic heart valve includes: an annular frame including a plurality of interconnected struts defining a plurality of rows of cells, the plurality of rows of cells arranged between an outflow end and an inflow end of the frame and including an upper row of cells arranged at the outflow end that are elongated in an axial direction relative to cells of remaining rows of cells of the plurality of rows of cells, the axial direction relative to a central longitudinal axis of the frame; a plurality of leaflets situated within the frame, each leaflet comprising opposing commissure tabs on opposite sides of the leaflet, each commissure tab being paired with an adjacent commissure tab of an adjacent leaflet to form a commissure; and at least one commissure support element comprising a coupling portion and two axially extending members that are radially offset from the coupling portion and are laterally spaced apart from one another to form an open, leaflet-receiving window configured to receive the commissure, where the coupling portion is configured to couple to an upper edge of two adjacent cells of the upper row of cells, the upper edge arranged at the outflow end of the frame, and where the leaflet-receiving window is spaced apart, in the axial direction, from the upper edge.

In another representative embodiments, a prosthetic heart valve includes an annular frame including a plurality of interconnected struts defining a plurality of rows of cells, the plurality of rows arranged between an outflow end and an inflow end of the frame and including an upper row of cells arranged at the outflow end that are elongated in an axial direction relative to cells of remaining rows of cells of the plurality of rows of cells, the axial direction relative to a central longitudinal axis of the frame, where struts defining the upper row of cells include a plurality of axial struts, each axial strut forming a common, axial side of two adjacent cells of the upper row of cells, and where a portion of the plurality of axial struts are window axial struts, each window axial strut including a closed strut window positioned a distance away from an upper end of the window axial strut, along a length of the window axial strut. The prosthetic heart valve further includes a plurality of leaflets situated within the frame, each leaflet comprising opposing commissure tabs on opposite sides of the leaflet, each commissure tab being paired with an adjacent commissure tab of an adjacent leaflet to form a commissure and at least one commissure support element comprising a coupling portion and two axially extending members that are radially offset from the coupling portion and are laterally spaced apart from one another to form an open, leaflet-receiving window configured to receive the commissure, where the coupling portion is configured to couple to the strut window.

In another representative embodiment, a prosthetic heart valve includes an annular frame including a plurality of interconnected struts defining a plurality of rows of cells, the plurality of rows arranged between an outflow end and an inflow end of the frame and including an upper row of cells arranged at the outflow end; and a plurality of leaflets situated within the frame, each leaflet comprising opposing commissure tabs on opposite sides of the leaflet, each commissure tab being paired with an adjacent commissure tab of an adjacent leaflet to form a commissure. Struts defining the upper row of cells further define a plurality of open commissure windows formed between adjacent cells of the upper row of cells, each commissure window configured to receive a commissure therein and formed by two axially extending window struts that are spaced apart from one another in a circumferential direction, wherein the commissure windows are offset axially toward an upstream/inflow end of the upper row of cells.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 shows a perspective view of a prosthetic heart valve, according to one embodiment.

FIG. 2 shows a perspective view of an exemplary frame of a prosthetic heart valve.

FIG. 3 shows a side view of the frame of FIG. 2, shown in a flattened configuration.

FIG. 4 shows a detail view of a portion of the frame of FIG. 3 with a pair of leaflets arranged in a window of the frame.

FIG. 5 shows a detail view of a portion of frame of a prosthetic heart valve including an open commissure window, according to one embodiment.

FIG. 6 shows a perspective view of an exemplary window fastener configured to be arranged within a portion of the open commissure window of FIG. 5.

FIG. 7 shows a top view of the window fastener of FIG. 6, arranged within the open commissure window of FIG. 5.

FIG. 8 shows a detail view of a portion of a frame of a prosthetic heat valve including an open commissure window, according to another embodiment.

FIG. 9 shows a detail view of the portion of the frame of FIG. 8 with a commissure arranged within the open commissure window.

FIG. 10 shows a detail view of a portion of a frame of a prosthetic heat valve including an open commissure window, according to another embodiment.

FIG. 11 shows a detail view of the portion of the frame of FIG. 10 with a commissure tab arranged within one open window portion of the open commissure window.

FIG. 12 shows a portion of an exemplary leaflet including an axial extension configured to be arranged within a portion of the open commissure window of FIG. 10.

FIG. 13 shows a sectional top view of an embodiment of a commissure arranged within the open commissure window of FIG. 10.

FIG. 14 shows a sectional top view of another embodiment of a commissure arranged within the open commissure window of FIG. 10.

FIG. 15 shows a detail view of a portion of a frame of a prosthetic heart valve including a bendable commissure window, according to an embodiment.

FIG. 16 shows a detail view of the portion of the frame of FIG. 15 with the commissure window bent over an axially extending strut of the frame and a commissure arranged within the commissure window.

FIG. 17 is a flow chart of a method for assembling a commissure within a bendable commissure window of a frame of a prosthetic heart valve and securing the bendable commissure window containing the commissure to the frame.

FIG. 18 shows an exemplary embodiment of a commissure support element configured to be coupled to a frame of a prosthetic heart valve and receive a commissure within a leaflet-receiving window of the commissure support element.

FIG. 19 shows a detail view of a portion of a frame of a prosthetic heart valve with the commissure support element of FIG. 18 coupled to an upper row of elongated cells of the frame.

FIG. 20 shows a commissure arranged within the commissure support element of FIG. 18 and the commissure support element coupled to the frame of FIG. 19.

FIG. 21 shows the commissure support element of FIG. 18 coupled to the upper row of elongated cells of the frame of FIG. 19, radially inward of an axial strut of the frame, the axial strut having a larger width than other axial struts of the frame.

FIG. 22 shows a detail view of a portion of a frame of a prosthetic heart valve including an axial strut with a strut window configured to receive a commissure support element.

FIG. 23 shows a detail view of the portion of the frame of FIG. 22 with an exemplary commissure support element coupled with the strut window.

FIG. 24 shows a detail view of the portion of the frame of FIG. 22 with the exemplary commissure support element coupled with the strut window and a commissure arranged within a leaflet-receiving window of the commissure support element.

FIG. 25 is a side view of an embodiment of a delivery apparatus configured to deliver and implant a radially expandable prosthetic heart valve at an implantation site.

### DETAILED DESCRIPTION

### General Considerations

For purposes of this description, certain aspects, advantages, and novel features of the embodiments of this disclosure are described herein. The described methods, systems, and apparatus should not be construed as limiting in any way. Instead, the present disclosure is directed toward all novel and non-obvious features and aspects of the various disclosed embodiments, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present, or problems be solved.

Features, integers, characteristics, compounds, chemical moieties, or groups described in conjunction with a particular aspect, embodiment or example of the disclosure are to be understood to be applicable to any other aspect, embodiment or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The disclosure is not restricted to the details of any foregoing embodiments. The disclosure extends to any novel one, or any novel combination, of the features disclosed in this specification (including any accompanying claims, abstract, and drawings), or to any novel one, or any novel combination, of the steps of any method or process so disclosed.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods, systems, and apparatus can be used in conjunction with other systems, methods, and apparatus.

As used herein, the terms “a,” “an,” and “at least one” encompass one or more of the specified element. That is, if two of a particular element are present, one of these elements is also present and thus “an” element is present. The terms “a plurality of” and “plural” mean two or more of the specified element.

As used herein, the term “and/or” used between the last two of a list of elements means any one or more of the listed elements. For example, the phrase “A, B, and/or C” means “A,” “B,” “C,” “A and B,” “A and C,” “B and C,” or “A, B, and C.”

As used herein, the term “coupled” generally means physically coupled or linked and does not exclude the presence of intermediate elements between the coupled items absent specific contrary language.

Directions and other relative references (e.g., inner, outer, upper, lower, etc.) may be used to facilitate discussion of the drawings and principles herein, but are not intended to be limiting. For example, certain terms may be used such as “inside,” “outside,”, “top,” “down,” “interior,” “exterior,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” part can become a “lower” part simply by turning the object over. Nevertheless, it is still the same part and the object remains the same. As used herein, “and/or” means “and” or “or,” as well as “and” and

In the context of the present application, the terms “lower” and “upper” are used interchangeably with the term's “inflow” and “outflow”, respectively. Thus, for example, the lower end of the valve is its inflow end and the upper end of the valve is its outflow end.

As used herein, with reference to the prosthetic heart valve and the delivery apparatus, “proximal” refers to a position, direction, or portion of a component that is closer to the user and/or a handle of the delivery apparatus that is outside the patient, while “distal” refers to a position, direction, or portion of a component that is further away from the user and/or the handle of the delivery apparatus and closer to the implantation site. The terms “longitudinal” and “axial” refer to an axis extending in the proximal and distal directions, unless otherwise expressly defined. Further, the term “radial” refers to a direction that is arranged perpendicular to the axis and points along a radius from a center of an object (where the axis is positioned at the center, such as the longitudinal axis of the prosthetic valve).

### Examples of the Disclosed Technology

Described herein are examples of prosthetic heart valves and methods for assembling prosthetic heart valves including an annular frame and a plurality of leaflets. The prosthetic heart valves may include an annular frame and a plurality of leaflets attached to the frame via commissures formed by joining pairs of adjacent ends (e.g., commissure tabs) of the leaflets. In some embodiments, the commissure may be at least partially formed outside the annular frame.

In some embodiments, the annular frame of the prosthetic heart valve can include a plurality of rows of cells formed by interconnected struts of the frame. The plurality of rows of cells can include an upper row of cells (e.g., arranged at an outflow end of the frame). In some embodiments, the cells of the upper row of cells are elongated in an axial direction relative to cells of remaining rows of cells of the frame.

In some embodiments, a portion of the struts of the annular frame forming axial sides of two adjacent elongated cells of the upper row of cells can form commissure windows that are adapted to receive a commissure and arrange the commissure within a lower region of the two adjacent elongated cells (e.g., within a region arranged farther away from the outflow end of the frame). In some embodiments, the frame can be configured such that the commissure windows are axially offset toward an inflow (e.g., upstream) end of the cells of the upper row of cells.

In other embodiments, a portion of the struts of the annular frame forming axial sides of two adjacent elongated cells of the upper row of cells can be adapted to receive a commissure support element, the commissure support element adapted to receive a commissure. Additionally, the portion of the struts and/or the commissure support element can be adapted to arrange the commissure within the lower region of the two adjacent elongated cells.

In this way, commissures and their associated leaflets may be arranged within lower regions of the upper rows of cells (e.g., offset from an outflow end of the frame and toward the inflow end), thereby leaving a relatively open space within an upper region of the upper rows of cells, proximate to the outflow end of the frame. This relative open space may provide increased blood flow through the valve and/or allow for passage of a re-access device, as described further herein.

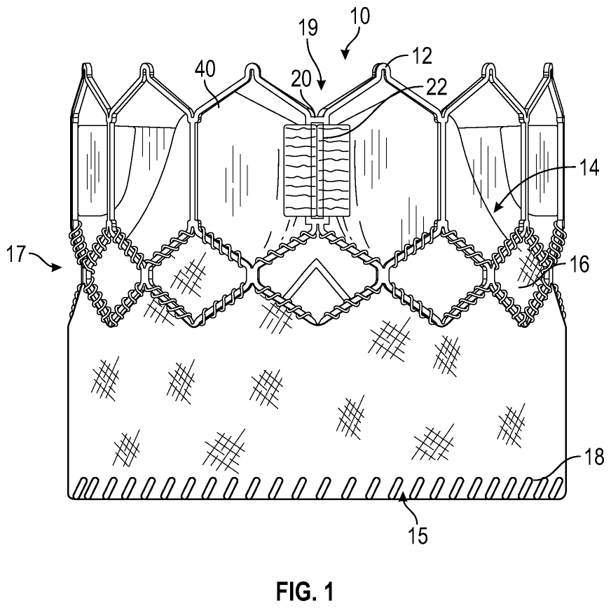

FIG. 1 shows a prosthetic heart valve 10, according to one embodiment. The illustrated prosthetic valve is adapted to be implanted in the native aortic annulus, although in other embodiments it can be adapted to be implanted in the other native annuluses of the heart (e.g., the pulmonary, mitral, and tricuspid valves). The prosthetic valve can also be adapted to be implanted in other tubular organs or passageways in the body. The prosthetic valve 10 can have four main components: a stent or frame 12, a valvular structure 14, an inner skirt 16, and a perivalvular outer sealing member or outer skirt 18. The prosthetic valve 10 can have an inflow end portion 15, an intermediate portion 17, and an outflow end portion 19.

The valvular structure 14 can comprise three leaflets 40, collectively forming a leaflet structure, which can be arranged to collapse in a tricuspid arrangement, although in other embodiments there can be greater or fewer number of leaflets (e.g., one or more leaflets 40). The leaflets 40 can be secured to one another at their adjacent sides to form commissures 22 of the leaflet structure 14. The lower edge of valvular structure 14 can have an undulating, curved scalloped shape and can be secured to the inner skirt 16 by sutures (not shown). In some embodiments, the leaflets 40 can be formed of pericardial tissue (e.g., bovine pericardial tissue), biocompatible synthetic materials, or various other suitable natural or synthetic materials as known in the art and described in U.S. Pat. No. 6,730,118, which is incorporated by reference herein.

The frame 12 can be formed with a plurality of circumferentially spaced slots, or commissure windows 20 that are adapted to mount the commissures 22 of the valvular structure 14 to the frame. The frame 12 can be made of any of various suitable plastically-expandable materials (e.g., stainless steel, etc.) or self-expanding materials (e.g., nickel titanium alloy (NiTi), such as nitinol), as known in the art. When constructed of a plastically-expandable material, the frame 12 (and thus the prosthetic valve 10) can be crimped to a radially collapsed configuration on a delivery catheter or apparatus and then expanded inside a patient by an inflatable balloon or equivalent expansion mechanism. When constructed of a self-expandable material, the frame 12 (and thus the prosthetic valve 10) can be crimped to a radially collapsed configuration and restrained in the collapsed configuration by insertion into a sheath or equivalent mechanism of a delivery catheter. Once inside the body, the prosthetic valve can be advanced from the delivery sheath, which allows the prosthetic valve to expand to its functional size.

Suitable plastically-expandable materials that can be used to form the frame 12 include, without limitation, stainless steel, a biocompatible, high-strength alloys (e.g., a cobalt-chromium or a nickel-cobalt-chromium alloys), polymers, or combinations thereof. In particular embodiments, frame 12 is made of a nickel-cobalt-chromium-molybdenum alloy, such as MP35N® alloy (SPS Technologies, Jenkintown, Pa.), which is equivalent to UNS R30035 alloy (covered by ASTM F562-02). MP35N® alloy/UNS R30035 alloy comprises 35% nickel, 35% cobalt, 20% chromium, and 10% molybdenum, by weight. Additional details regarding the prosthetic valve 10 and its various components are described in WIPO Patent Application Publication No. WO 2018/222799, which is incorporated herein by reference.

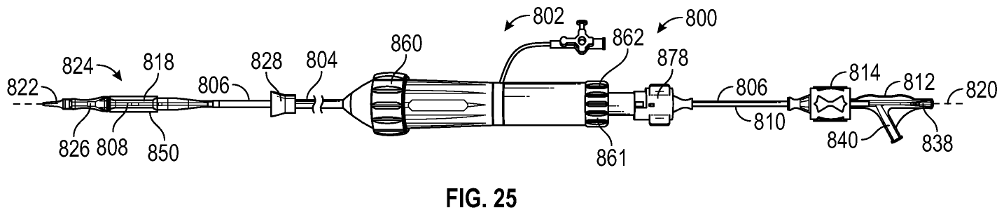

FIG. 25 shows a delivery apparatus 800, according to an embodiment, that can be used to implant an expandable prosthetic heart valve (e.g., prosthetic heart valve 10 of FIG. 1 or any of the other prosthetic heart valves described herein). In some embodiments, the delivery apparatus 800 is specifically adapted for use in introducing a prosthetic valve into a heart.

The delivery apparatus 800 in the illustrated embodiment of FIG. 25 is a balloon catheter comprising a handle 802 and a steerable, outer shaft 804 extending distally from the handle 802. The delivery apparatus 800 can further comprise an intermediate shaft 806 (which also may be referred to as a balloon shaft) that extends proximally from the handle 802 and distally from the handle 802, the portion extending distally from the handle 802 also extending coaxially through the outer shaft 804. Additionally, the delivery apparatus 800 can further comprise an inner shaft 808 extending distally from the handle 802 coaxially through the intermediate shaft 806 and the outer shaft 804 and proximally from the handle 802 coaxially through the intermediate shaft 806.

The outer shaft 804 and the intermediate shaft 806 can be configured to translate (e.g., move) longitudinally, along a central longitudinal axis 820 of the delivery apparatus 800, relative to one another to facilitate delivery and positioning of a prosthetic valve at an implantation site in a patient's body.

The intermediate shaft 806 can include a proximal end portion 810 that extends proximally from a proximal end of the handle 802, to an adaptor 812. A rotatable knob 814 can be mounted on the proximal end portion 810 and can be configured to rotate the intermediate shaft 806 around the central longitudinal axis 820 and relative to the outer shaft 804.

The adaptor 812 can include a first port 838 configured to receive a guidewire therethrough and a second port 840 configured to receive fluid (e.g., inflation fluid) from a fluid source. The second port 840 can be fluidly coupled to an inner lumen of the intermediate shaft 806.

The intermediate shaft 806 can further include a distal end portion that extends distally beyond a distal end of the outer shaft 804 when a distal end of the outer shaft 804 is positioned away from an inflatable balloon 818 of the delivery apparatus 800. A distal end portion of the inner shaft 808 can extend distally beyond the distal end portion of the intermediate shaft 806.

The balloon 818 can be coupled to the distal end portion of the intermediate shaft 806.

In some embodiments, a distal end of the balloon 818 can be coupled to a distal end of the delivery apparatus 800, such as to a nose cone 822 (as shown in FIG. 25), or to an alternate component at the distal end of the delivery apparatus 800 (e.g., a distal shoulder). An intermediate portion of the balloon 818 can overlay a valve mounting portion 824 of a distal end portion of the delivery apparatus 800 and a distal end portion of the balloon 818 can overly a distal shoulder 826 of the delivery apparatus 800. The valve mounting portion 824 and the intermediate portion of the balloon 818 can be configured to receive a prosthetic heart valve in a radially compressed state. For example, as shown schematically in FIG. 25, a prosthetic heart valve 850 (which can be one of the prosthetic valves described herein) can be mounted around the balloon 818, at the valve mounting portion 824 of the delivery apparatus 800.

The balloon shoulder assembly, including the distal shoulder 826, is configured to maintain the prosthetic heart valve 850 (or other medical device) at a fixed position on the balloon 818 during delivery through the patient's vasculature.

The outer shaft 804 can include a distal tip portion 828 mounted on its distal end. The outer shaft 804 and the intermediate shaft 806 can be translated axially relative to one another to position the distal tip portion 828 adjacent to a proximal end of the valve mounting portion 824, when the prosthetic valve 850 is mounted in the radially compressed state on the valve mounting portion 824 (as shown in FIG. 25) and during delivery of the prosthetic valve to the target implantation site. As such, the distal tip portion 828 can be configured to resist movement of the prosthetic valve 850 relative to the balloon 818 proximally, in the axial direction, relative to the balloon 818, when the distal tip portion 828 is arranged adjacent to a proximal side of the valve mounting portion 824.

An annular space can be defined between an outer surface of the inner shaft 808 and an inner surface of the intermediate shaft 806 and can be configured to receive fluid from a fluid source via the second port 840 of the adaptor 812. The annular space can be fluidly coupled to a fluid passageway formed between the outer surface of the distal end portion of the inner shaft 808 and an inner surface of the balloon 818. As such, fluid from the fluid source can flow to the fluid passageway from the annular space to inflate the balloon 818 and radially expand and deploy the prosthetic valve 850.

An inner lumen of the inner shaft can be configured to receive a guidewire therethrough, for navigating the distal end portion of the delivery apparatus 800 to the target implantation site.

The handle 802 can include a steering mechanism configured to adjust the curvature of the distal end portion of the delivery apparatus 800. In the illustrated embodiment, for example, the handle 802 includes an adjustment member, such as the illustrated rotatable knob 860, which in turn is operatively coupled to the proximal end portion of a pull wire. The pull wire can extend distally from the handle 802 through the outer shaft 804 and has a distal end portion affixed to the outer shaft 804 at or near the distal end of the outer shaft 804. Rotating the knob 860 can increase or decrease the tension in the pull wire, thereby adjusting the curvature of the distal end portion of the delivery apparatus 800. Further details on steering or flex mechanisms for the delivery apparatus can be found in U.S. Pat. No. 9,339,384, which is incorporated by reference herein.

The handle 802 can further include an adjustment mechanism 861 including an adjustment member, such as the illustrated rotatable knob 862, and an associated locking mechanism including another adjustment member, configured as a rotatable knob 878. The adjustment mechanism 861 is configured to adjust the axial position of the intermediate shaft 806 relative to the outer shaft 804 (e.g., for fine positioning at the implantation site). Further details on the delivery apparatus 800 can be found in U.S. Provisional Application Nos. 63/069,567 and 63/138,890, which are incorporated by reference herein.

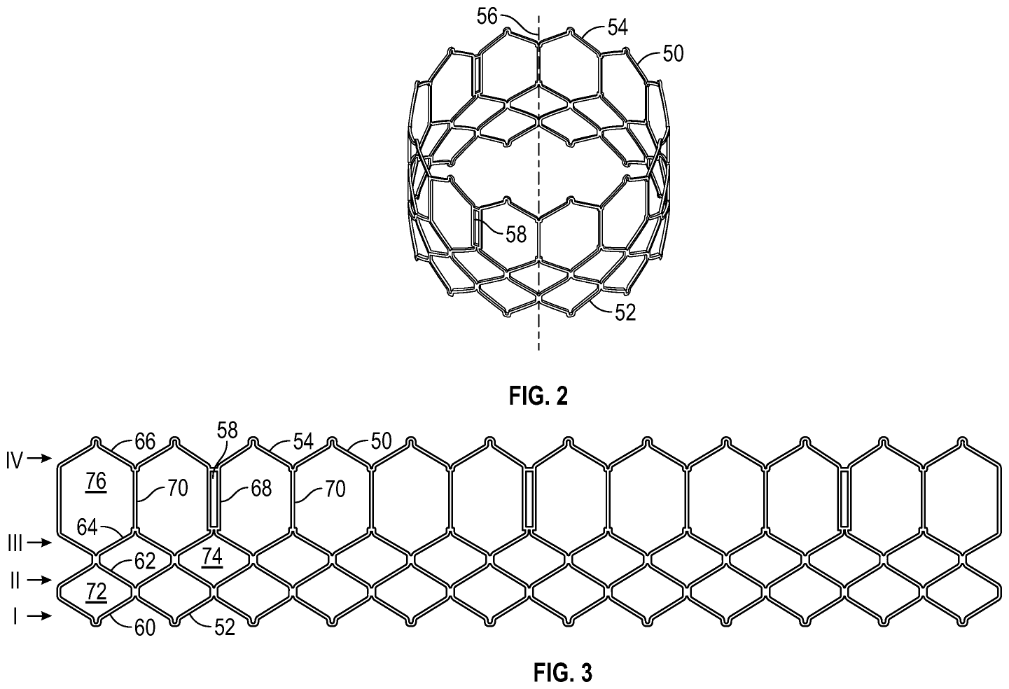

FIGS. 2-3 show a bare frame 50 of a prosthetic heart valve, which, in some embodiments, can be the frame 12 of the prosthetic valve 10 shown in FIG. 1. The frame 50 has an inflow end 52, an outflow end 54, and a central longitudinal axis 56 extending from the inflow end 52 to the outflow end 54 (e.g. in an axial direction). The frame 50 can be made of any of the materials described above with reference to FIG. 1. As described above with reference to FIG. 1, the frame 50 can comprise a plurality of commissure windows (e.g., open windows) 58 spaced apart from one another, in a circumferential direction (e.g., around a circumference of the frame 50), which are each adapted to receive a pair of commissure tabs of a pair of leaflets arranged into a commissure.

As shown in FIG. 3, the frame 50 comprises a first, lower row I of angled struts 60 arranged end-to-end and extending circumferentially at the inflow end of the frame; a second row II of circumferentially extending, angled struts 62; a third row III of circumferentially extending, angled struts 64; and a fourth row IV of circumferentially extending, angled struts 66 at the outflow end of the frame 12. The fourth row IV of angled struts 66 can be connected to the third row III of angled struts 64 by a plurality of axially extending window frame (or strut) portions 68 (which define commissure windows 58) and a plurality of axially extending struts 70. Each axial strut 70 and each frame portion 68 extends from a location defined by the convergence of the lower ends (e.g., ends arranged inward of and farthest away from the outflow end 54) of two angled struts 66 (also referred to herein as an upper strut junction or upper elongated strut junction) to another location defined by the convergence of the upper ends (e.g., ends arranged closer to the outflow end 54) of two angled struts 64 (also referred to herein as a lower strut junction or lower elongate strut junction).

As used herein with reference to the strut ends, cells, and other frame components, “upper” and “lower” may be relative to the shown, vertical arrangement of the frame. For example, “upper” components may be arranged closer to the outflow end than the inflow end of the frame while “lower” components may be arranged closer to the inflow end than the outflow end of the frame. Thus, in some embodiments, an “upper end” of a strut or commissure window frame portion may be referred to as an outflow-facing end and a “lower end” of the strut or commissure window frame portion may be referred to as an inflow-facing end, relative to the inflow and outflow ends of the frame, as described herein. In yet other embodiments, an “upper” end, junction, or component may be referred to as a “proximal” end, junction, or component and a “lower end, junction, or component may be referred to as a “distal” end, junction or component.

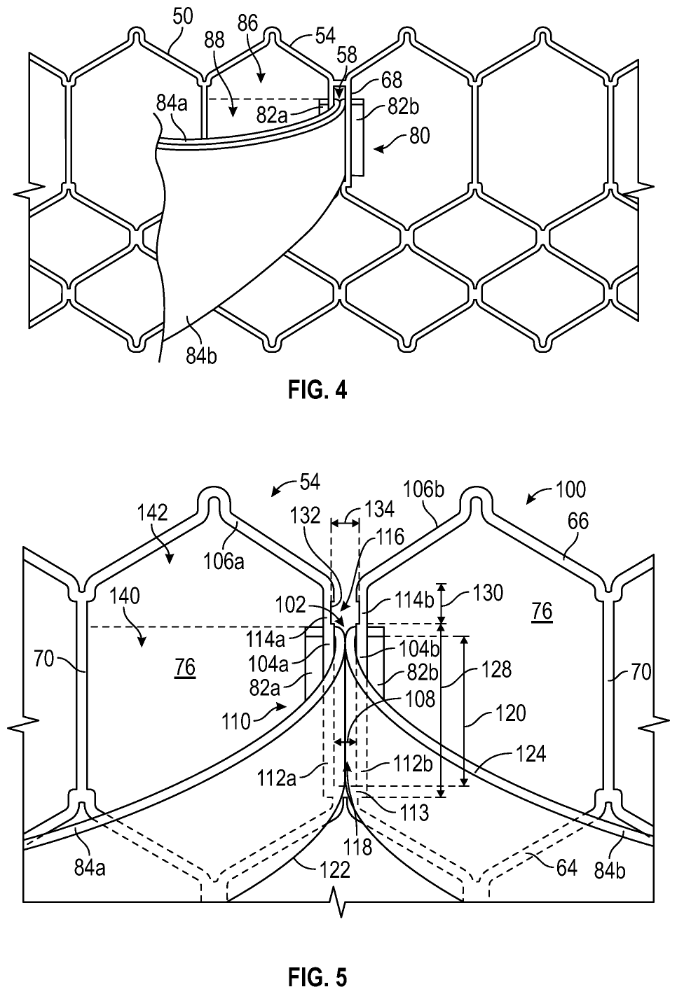

Each commissure window frame portion 68 mounts a respective commissure of the leaflet structure (e.g., as shown in FIG. 1 and FIG. 4). As can be seen in FIG. 3, each frame portion 68 is secured at its upper and lower ends to the adjacent rows of struts to provide a robust configuration that may enhance fatigue resistance under cyclic loading of the valve compared to known cantilevered struts for supporting the commissures of the leaflet structure.

The struts and frame portions of the frame 50 collectively define a plurality of open cells of the frame. At the inflow end 52 of the frame 50, struts 60 and struts 62 define a lower row of cells defining openings 72. The second and third rows of struts 62 and 64, respectively, define an intermediate row of cells defining openings 74. The third and fourth rows of struts 64 and 66, along with frame portions 68 and struts 70, define an upper row of cells defining openings 76. The openings 76 are relatively large (e.g., larger relative to the openings 72 and 74) and can be referred to as elongated openings 76.

In some embodiments, as used herein, the struts 66 may be referred to as upper angled struts 66 of the elongated cells of the upper row of cells and the struts 64 may be referred to as lower angled struts 64 of the elongated cells of the upper row of cells.

Though the embodiment of FIG. 3 shows three rows of cells, with the upper row of cells being elongated, in alternate embodiments, the frame 50 may have a different number of rows of non-elongated cells. For example, in alternate embodiments, the frame 50 may include the upper row of elongated cells, defining openings 76, and a single row of cells with smaller, not elongated openings (e.g., defining openings 74). In yet another embodiment, the frame 50 may include the upper row of elongated cells, defining openings 76, and three additional rows of cells with smaller, not elongated openings.

In some embodiments, the frame may be a smaller diameter frame for a smaller diameter prosthetic heart valve. For example, conventional transcatheter heart valves may have expansion diameters in a range of 23-29 mm. As an example, smaller diameter transcatheter heart valves can have a diameter of approximately 20 mm. In some embodiments, the smaller diameter valves can have a diameter of less than 22 mm. In other embodiments, the smaller diameter valves can have a diameter in a range of 19-21 mm. However, these smaller diameter valves may be used less frequently due to a variety of challenges that arise from their naturally smaller cell size and leaflet arrangement within the frame. As one example, the smaller sized cells of the smaller diameter valve may decrease or interfere with blood flow into the coronary ostia following a valve-in-valve (e.g., prosthetic valve-in-prosthetic valve) procedure. As another example, the smaller sized cells may inhibit the passage therethrough of optional coronary re-access devices, at a later time, following valve implantation.

To at least partially address these issues, the upper row of cells (e.g., cells defining openings 76 in FIG. 3), arranged proximate to the outflow end of the frame can be elongated relative to the other rows of cells of the frame, as shown in FIGS. 2-3 and 4 (as described further below). However, the leaflet assembly of the prosthetic valve, including commissures attached at the commissure windows 58 (e.g., as shown in FIG. 1), may at least partially block the openings of the upper rows of cells, thereby reducing access for blood flow and coronary re-access devices.

Thus, it may be desirable to have commissure attachment configurations that retain the leaflets of the prosthetic valve in a position within the frame that maintains a relatively open cell area within the upper cells, proximate to the outflow end of the frame, that is sufficient to allow blood flow to the coronary ostia, and passage of a coronary re-access device, once the prosthetic valve is implanted.

For example, as shown in FIG. 4, a commissure 80 comprising a pair of commissure tabs 82a and 82b of respective leaflets 84a and 84b can be arranged within a lower portion of the commissure window 58. Specifically, FIG. 4 shows a detail view of a portion of the frame 50 (as shown in its entirety in FIG. 3), including the commissure window 58 and the pair of commissure tabs 82a and 82b extending therethrough. As described above, it is desirable to maintain the commissure 80 within the lower portion of the commissure window 58, such that the commissure 80 is arranged adjacent to a lower edge of the window frame portions 68 (e.g., toward an inflow or upstream end of the upper cells). As a result, the commissure 80 and the leaflets 84a and 84b are axially offset away from the outflow end of the valve and maintained within a lower region 88 of the upper row of cells, thereby maintaining a relatively open space within an upper region 86 of the upper row of cells. This can result in a more open cell area within the upper cells, proximate to the outflow end 54 of the frame 50.

Thus, it is desirable to configure the frame of the prosthetic valve and/or commissure support (or attachment) elements that are attachable to the frame such that they maintain the commissures, and their corresponding leaflets, within lower regions (or portions) of the upper, elongated cells. Additionally, the frame and/or commissure support elements can be configured such that the commissures can be pre-assembled outside the frame and then inserted (e.g., slid) into the frame and/or commissure support element (e.g., from above or below due to an at least partially open commissure window in the frame and/or commissure support element). This configuration and assembly method may decrease assembly time for the prosthetic valve and increase the overall ease of manufacturing the prosthetic valve.

FIGS. 5, 8-11, 13-16, and 19-24 show different embodiments of a frame of a prosthetic heart valve having elongated cells at an outflow end of the frame where at least a portion of a plurality of elongated struts forming the elongated cells are configured to receive commissures and/or commissure support elements coupled with commissures. The portion of the plurality of elongated struts can be configured to maintain the commissures within a lower portion of the elongated cells that is arranged away from the outflow end of the frame, thereby maintaining an opposite, upper portion of the elongated cells open and unblocked by leaflets of the prosthetic heart valve. Additionally, the portion of the plurality of elongated struts can be configured to receive the commissure support elements or pre-assembled commissures.

The prosthetic heart valve frames shown in FIGS. 5, 8-11, 13-16, and 19-24 may be similar to frame 50 shown in FIGS. 2-3, and thus, components of the frames shown in FIGS. 5, 8-11, 13-16, and 19-24 that are identical to components of frame 50 have been labeled the same. For example, the different frame embodiments shown in FIGS. 5, 8-11, 13-16, and 19-24 may have commissure window frame portions and/or axial struts, which at least partially form the elongated cells at the outflow end, that are modified from frame 50, while a remainder of the frame remains similar to that of frame 50 (e.g., the struts forming the smaller, lower rows of cells). Thus, even though FIGS. 5, 8-11, 13-16, and 19-24 may only show a portion of a frame of a prosthetic heart valve containing commissure window frame portions or struts adapted to receive a commissure or commissure support element, the omitted portions of the frame can be similar to the corresponding portions of frame 50 of FIGS. 2-3.

FIG. 5 shows a first embodiment of a frame 100 having elongated cells at an outflow end 54 of the frame 100 and a plurality of open commissure windows 102 (only one shown in FIG. 5) formed by axially extending window struts 104a and 104b of the elongated cells, each open commissure window 102 configured to receive a commissure within a lower region (or portion) of the open commissure window 102. The open commissure windows 102 are each formed by a gap region between axially extending window struts of adjacent elongated cells. Further, each open commissure window 102 includes a lower region 118 configured to receive commissure tabs of a commissure and an upper region 116 arranged proximate to the outflow end 54 of the frame 100.

More specifically, as shown in FIG. 5, the open commissure window 102 is formed by and between a first axially extending window strut 104a of a first elongated cell 106a and a second axially extending window strut 104b of a second elongated cell 106b, the first and second elongated cells 106a and 106b arranged adjacent to one another within the frame 100, in a circumferential direction. The first axially extending window strut 104a and the second axially extending window strut 104b are spaced apart from one another, in the circumferential direction, by a distance (e.g., gap) 108. The distance 108 can be selected for receiving commissure tabs 82a and 82b of the commissure 110 therein (e.g., may be the same or slightly smaller or bigger than a width of two commissure tabs).

As shown in FIG. 5, the open commissure window 102 is open ended at its upper (also referred to as proximal) end (e.g., the end closest to the outflow end 54 of the frame 100). As a result, during assembly of the prosthetic heart valve, the commissure 110, including the secured together commissure tabs 82a and 82b, can be slid into the open commissure window 102 (e.g., the gap 108 formed between the first and second axially extending window struts 104a and 104b).

For example, the commissure 110 can be formed outside of the frame 100 by connecting the commissure tabs (e.g., commissure tabs 82a and 82b) of two adjacent leaflets (e.g., leaflets 84a and 84b) to one another. In some embodiments, the commissure tabs of the commissure 110 can be connected to one another via one or more sutures. In some embodiments, each of the commissure tabs of the commissure 110 can be folded over and away from the adjacent leaflet (e.g., at approximately 90 degrees) and a reinforcing member can be positioned between the folded over (e.g., bent) corners of the commissure tabs. The reinforcing member can be secured to the commissure tabs via one or more fasteners (e.g., sutures). In some embodiments, the reinforcing member can be referred to as a wedge member. The reinforcing member can have a width (extending in the circumferential direction) that is large enough to prevent the commissure tabs from being pulled back through the open commissure window 102, after the commissure 110 is inserting into the open commissure window 102 and the leaflets are arranged within the frame 100. It should be noted that, in some embodiments, the other commissures described herein can be formed in a similar way to commissure 110, as described above.

Each of the axially extending window struts 104a and 104b comprises a respective lower (or distal) window strut portion 112a and 112b and a respective upper (or proximal) window strut portion 114a and 114b. Thus, the commissure window lower region 118 is defined between the lower window strut portions 114a and 114b and the commissure window upper region 116 is defined between the upper window strut portions 114a and 114b.

Additionally, the commissure window 102 is closed at its lower (e.g., distal) end. For example, the commissure window 102 can be closed at its lower end by a lateral window strut portion 113 connecting lower ends of the lower window strut portions 112a and 112b of the axially extending window struts 104a and 104b together.

As shown in FIG. 5, the lateral window strut portion 113 further connects to angled struts 64 which define a portion of a cell of a second row of cells arranged adjacent to the upper row of elongated cells.

In this way, the commissure window lower region 118 is closed at its lower end and lower edges of the commissure tabs 82a and 82b can be positioned against the lateral window strut portion 113.

As shown in FIG. 5, a height 120 of each of the commissure tabs 82a and 82b, the height 120 extending between a scallop-line edge 122 and an upper edge 124 of each commissure tab, can closely match (e.g., be the same or slightly smaller than) a height of the commissure window lower region 118 (same as the length 128 of the lower window strut portions 112a and 112b, as described below).

Since the height (e.g., length 128) of the commissure window lower region 118 is smaller than a total height of the open commissure window 102, the leaflets 84a and 84b of the commissure tabs 82a and 82b arranged therein do no block at least an upper portion (e.g., region) 142 of the openings 76 of the elongated cells 106a and 106b. As a result, the openings 76 have an open area that may be large enough to ensure adequate blood flow to the coronary ostia once the prosthetic heart valve is implanted, as well as allowing re-access devices to pass therethrough.

In some embodiments, the length 128 of the lower window strut portions 112a and 112b can be longer than a length 130 of the upper window strut portions 114a and 114b. In some embodiments, the length 128 of the lower window strut portions 112a and 112b is in a range of 4-6 mm and the length 130 of the upper window strut portions 114a and 114b is in a range of 1 to 2 mm. In some embodiments, the length 128 of the lower window strut portions 112a and 112b is in a range of 4.4-5.5 mm and the length 130 of the upper window strut portions 114a and 114b is in a range of 1.25 to 1.75 mm. In some embodiments, the length 128 of the lower window strut portions 112a and 112b is approximately 5 mm and the length 130 of the upper window strut portions 114a and 114b is approximately 1.5 mm.

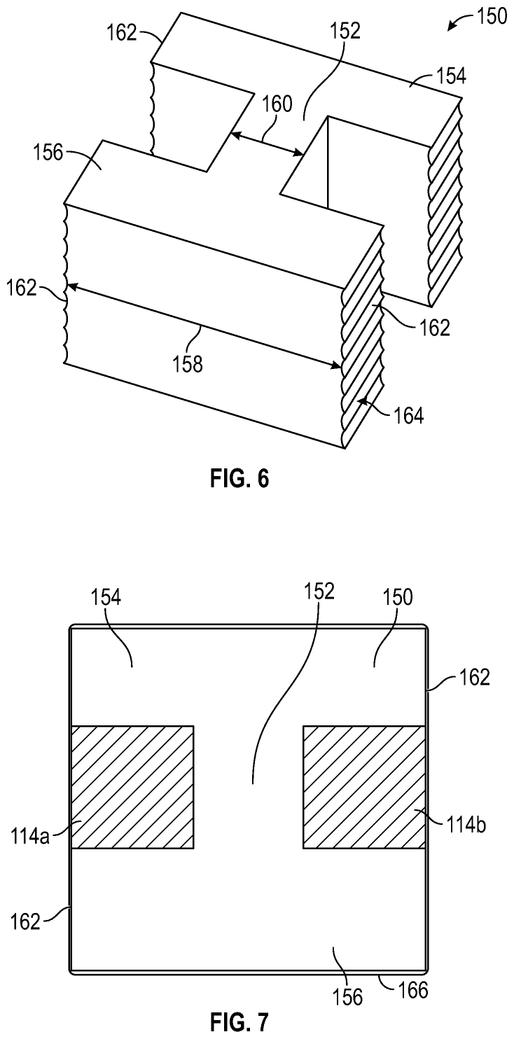

In some embodiments, the upper window strut portions 114a and 114b each include a recessed portion 132 that results in a narrowed portion of the respective upper window strut portion. A length of each recessed portion 132 can be shorter than the length 130 of the respective upper window strut portion 114a or 114b. As explained further below with reference to FIGS. 6 and 7, the recessed portions 132 of the upper window strut portions 114a and 114b can be shaped to receive a window fastener (e.g., spacer).

In some embodiments, the length 128 of the lower window strut portions 112a and 112b is chosen so as to closely match the height 120 of the commissure tabs 82a and 82b.

In some embodiments, the length 130 of the upper window strut portions 114a and 114b is chosen so as to retain an open cell area within the elongated cells 106a and 106b once the commissures 80 are assembled within respective open commissure windows 102, which is large enough to ensure adequate blood flow to the coronary ostia once implanted, as well as allow re-access devices to pass therethrough.

After the prosthetic heart valve is implanted, during operation of the valve, the leaflets are movable from an open state during the systolic phase to a closed state during the diastolic phase. The constant cyclical movement of the leaflets may exert lateral forces (e.g., in the circumferential direction) on the axially extending window struts 104a and 104b that may cause them to extend away from each other. Thus, in some embodiments, a commissure window fastener can be inserted between the axially extending window struts of each open commissure window (e.g., window 102). The commissure window fastener can be configured to secure the axially extending window struts of an open commissure window together to prevent lateral movement of the axially extending window struts relative to one another. In some embodiments, the commissure window fastener may be referred to as a spacer since it is adapted to maintain a relatively constant distance, in the circumferential (e.g., lateral) direction, between the axially extending window struts of an open commissure window.

FIGS. 6 and 7 show an exemplary embodiment of such a commissure window fastener 150. Specifically, FIG. 6 shows a perspective view of the window fastener 150 and FIG. 7 shows a top view of the window fastener 150 coupled within the open commissure window 102.

As shown in FIGS. 6 and 7, the window fastener 150 has an H (or I, in some embodiments) shape. The window fastener 150 comprises a middle portion 152 and two opposing end portions 154 and 156, the middle portion 152 extending between the two end portions 154 and 156. The two end portions 154 and 156 have a width 158 that is wider than a width 160 of the middle portion 152.

FIG. 7 shows a top view of the window fastener 150 inserted into the upper region 116 of the commissure window 102. Specifically, in some embodiments, the middle portion 152 can be slid from the open end of the commissure window 102 (proximate to the upper region 116) and downwards into upper region 116. As such, the middle portion 152 is arranged between the upper window strut portions 114a and 114b, while the end portions 154 and 156 are positioned radially outward and radially inward, respectively, of the frame 100 (e.g., of the struts of the frame). As used herein, a radial direction is relative to the central longitudinal axis of the annular frame 100. In this way, a first end portion 154 extends circumferentially along and between radially outward facing sides of the upper window strut portions 114a and 114b and a second end portion 156 extends circumferentially along and between radially inward facing sides of the upper window strut portions 114a and 114b.

As shown in FIG. 5 and introduced above, in some embodiments, the upper window strut portions 114a and 114b can each include a recessed portion 132, configured to accept the middle portion 152 of the window fastener 150.

Thus, the window fastener 150 can be slid into the window 102 from above (e.g., the outflow end of the frame), slightly extending (e.g., pushing) the upper window strut portions 114a and 114b away from each other during an initial phase of insertion, until the middle portion 152 snaps into place within the recessed portions 132 of the upper window strut portions 114a and 114b.

In some embodiments, the width 160 of the middle portion 152 may be approximately the same as a resting distance 134 (e.g., not under tension or load) between the two upper window strut portions 114a and 114b of the window 102, in a region of the recessed portions 132.

As shown in FIG. 6, in some embodiments, the lateral edges 162 of the two end portions 154 and 156 comprise a plurality of indentations (e.g., dents) 164, configured to accommodate and support fasteners (e.g., sutures) extending around and along the lateral edges 162. For example, after the window fastener 150 is positioned in place within the upper region 116 of the window 102, a suture 166 can loop around the end portions 154 and 156, thereby securing the upper window strut portions 114a and 114b tightly pressed against the window fastener 150.

In this way, the window fastener 150 can maintain a relatively constant distance between the axially extending window struts 104a and 104b, at all times during operation of the prosthetic heart valve. As a result, compression that may otherwise be applied to the commissure tabs arranged within the window 102 may be reduced or eliminated.

The frame 100 described above with reference to FIG. 5 provides commissure windows with commissure-receiving portions (e.g., lower region 118) that are configured to maintain the commissures within a lower portion 140 of the elongated cells (e.g., elongated cells 106a and 106b), the lower portion 140 arranged away from the outflow end 54 of the frame 200. As a result, an opposite, upper portion 142 of the elongated cells is maintained open and unblocked by leaflets of the prosthetic heart valve (as shown in FIG. 5).

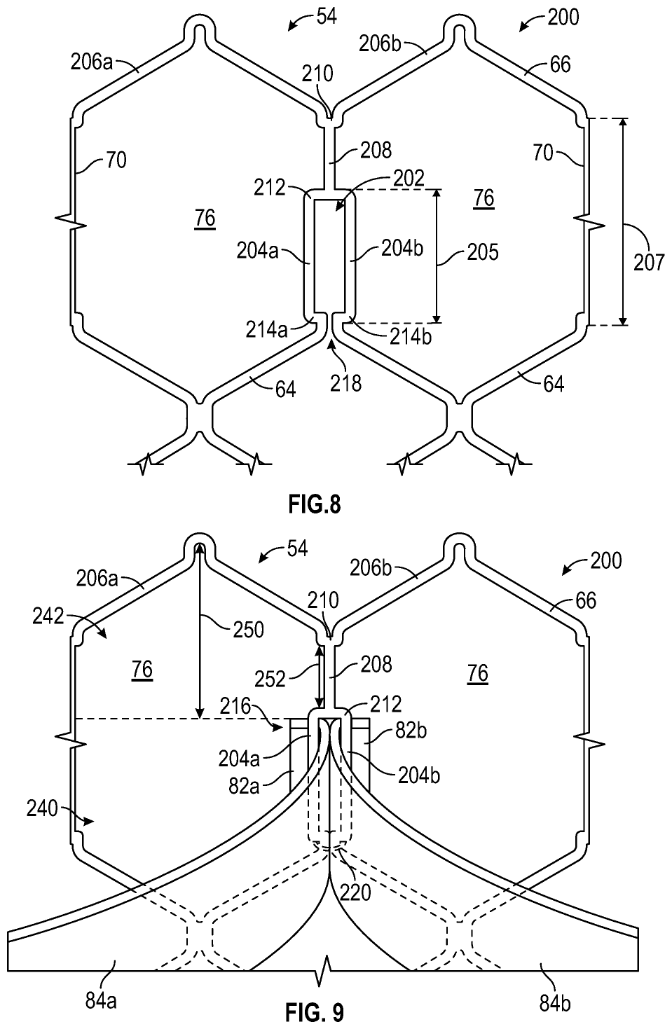

In another embodiment, as shown in FIGS. 8 and 9, a frame 200 having elongated cells at an outflow end 54 of the frame 200 can have a plurality of open commissure windows 202 (only one shown in FIGS. 8 and 9) defined between parallel, axially extending window struts of an axially extending strut of the elongated cells, the axially extending window struts extending from an upper axial strut portion of the axially extending strut. Each commissure window 202 can be open at a lower end of the commissure window 202 and arranged within a lower portion of two adjacent elongated cells. For example, the axially extending window struts can include lower (e.g., distal) clamping portions defining a bottom opening therebetween through which the commissure may slide into the commissure window 202 and that are configured to be clamped together to retain the commissure therein. In this way, the commissure window 202, configured to receive a commissure therein (as shown in FIG. 9), is positioned at a lower position within the elongated cells (e.g., away from the outflow end), thereby maintaining an open region for blood flow and device access at an upper portion of the elongated cells.

More specifically, as shown in FIGS. 8 and 9, an exemplary commissure window 202 is formed and defined between a first axially extending window strut 204a and a second axially extending window strut 204b. As shown in FIG. 8, a length 205 of the axially extending window struts 204a and 204b is shorter than a length 207 of an axial strut 70 of the elongated cells 206a and 206b of the frame 200 (the axial strut 70 not including a commissure window). The axially extending window struts 204a and 204b are integrally formed with the frame 200, each extending from an upper (e.g., proximal) axial strut 208. Together, the axially extending window struts 204a and 204b and the upper axial strut 208 can form an axially extending frame portion defining an axial side of each of the adjacent elongated cells 206a and 206b.

In some embodiments, a thickness or width of the upper axial strut 208, defined in the circumferential direction and perpendicular to the central longitudinal axis of the frame, can be the same as a thickness or width of the axially extending window struts 204a and 204b and/or additional struts of the frame (e.g., angled struts 66).

In some embodiments, the thickness or width of the upper axial strut 208 can be larger (e.g., thicker) than the thickness or width of the axially extending window struts 204a and 204b and/or additional struts of the frame (e.g., angled struts 66).

In some embodiments, the upper axial strut 208 can include additional geometrical features, such as one or more holes or notches along its length.

The upper axial strut 208 is arranged between an upper elongated strut junction 210 (e.g., the junction between two angled struts 66 and the upper axial strut 208) and an upper edge 212 of the commissure window 202. The upper edge 212 is arranged substantially perpendicular to the upper axial strut 208 and laterally (e.g., circumferentially) offsets the axially extending window struts 204a and 204b from each other. Said another way, the upper edge 212 extends between, in the circumferential (or lateral) direction, upper ends of each of the axially extending window struts 204a and 204b.

As shown in FIG. 8, what would be a lower elongated strut junction is replaced with an open, lower end of the commissure window 202.

For example, in some embodiments, each axially extending window strut 204a and 204b includes a lower clamping portion 214a and 214b, respectively, formed by a lower, bent (e.g., angled) portion of the respective axially extending window strut 204a and 204b. As shown in FIGS. 8 and 9, the bends of the lower clamping portions 214a and 214b are angled toward each other. Thus, as shown in FIGS. 8 and 9, together, the lower clamping portions 214a and 214b can form a neck region at the lower end of the open commissure window 202.

As shown in FIG. 9, commissure tabs 82a and 82b of a commissure 216 can extend through the open commissure window 202 to form a commissure assembly (e.g., commissure assembled to the frame 200). For example, in some embodiments, commissure tabs 82a and 82b of leaflets 84a and 84b can be inserted into the commissure window 202, through the opening 218 defined between the lower clamping portions 214a and 214b.

In some embodiments, the axially extending window struts 204a and 204b and their respective lower clamping portions 214a and 214b may be flexed or resiliently bent sideways (e.g., laterally outward and away from one another) during leaflet insertion.

In some embodiments, a suture 220 can be wrapped or looped around the lower clamping portions 214a and 214b (e.g., wrapped or looped around outer bends in each of the lower clamping portions), once the commissure tabs are positioned within the commissure window 202, so as to clamp the lower ends of the axially extending window struts 204a and 204b to each other, and to prevent the commissure tabs 82a and 82b from sliding axially out of the commissure window 202. In alternate embodiments, alternative fasteners or clamping means (e.g., bands, strings, ties, or the like) can be used to clamp the lower clamping portions 214a and 214b together.

In some embodiments, the lower clamping portions 214a and 214b can each include an angled, lower portion, and a relatively straight, horizontal upper portion. The angled portion may assist in sliding of the commissure tabs 82a and 82b into the commissure window 202 from the bottom opening, upward (toward the outflow end 54). The horizontal portions may serve as a lower (e.g., bottom) base supporting the commissure tabs 82a and 82b retained within the commissure window 202.

In alternate embodiments, the lower portion of the lower clamping portions 214a and 214b may not be angled, but may instead be relatively straight and arranged perpendicular to the horizontal, upper portion.

The upper edge 212 can be configured to reduce or prevent axial displacement of the commissure 216 upwards, in the axial direction toward the outflow end 54. In some embodiments, a wedge member (not shown) can be inserted between the commissure 216 (e.g., upper edges of the commissure tabs) and the upper edge 212, so as to further reduce or prevent undesirable axial movement of the commissure 216 within the commissure window 202. The wedge member can be a fabric or polymeric material having a thickness that is selected to be large enough to be positioned between and firmly against the upper edge 212 and upper edges of the commissure tabs 82a and 82b.

In this way, the open (e.g., open-ended at one end) windows 202 of the frame 200 of FIGS. 8 and 9 provide a way of inserting and coupling commissures of a leaflet assembly to a frame having elongated upper cells, thereby increasing an ease of manufacturing the prosthetic heart valve. Further, by having open windows 202 with lower clamping portions that may be clamped together to secure the commissures within respective windows 202, the commissures may be retained tightly within their respective window 202 at all times, including during transitions between crimped and expanded states of the prosthetic heart valve frame, and during diastole (during operating of the valve, in vivo).

Further, in some embodiments, the axial position (along the central longitudinal axis of the frame 200) of the upper edge 212, or the axial distance between the upper edge 212 and the outflow end of the frame 200, can be selected such that the leaflets are maintained within a lower portion 240 of the elongated cells and an upper portion 242 of the elongated cells remain unblocked by the leaflets. In this way, the commissure window 202, and thus the leaflets, are offset axially toward an inflow end (which can also be referred to as an upstream end) of the upper row of cells (e.g., cells 206a and 206b). As a result, during operation of the valve, adequate blood flow and/or access via a re-access device may be possible through the upper portions 242 of the elongated cells.

For example, in some embodiments, a length 252 of the upper axial strut 208 can be selected such that an axial distance 250 between the outflow end 54 (e.g., an apex or outflow end portion of struts 66 at the outflow end 54) of the frame 200 and outflow edges of the leaflets 84a and 84b at the commissure (or axial position of the upper edge 212, as denoted by the dashed line in FIG. 9) is within a selected range. In some embodiments, the selected range of the axial distance 250 is in a range of 2-6 mm, 2-4 mm, or 2-3 mm. In some embodiments, the selected range of the axial distance 250 is in a range of 20-50%, 25-45%, or 30-40% of the total axial distance (or height) of the elongated cells 206a and 206b. In some embodiments, the length 252 of the upper axial strut 208 can be in a range of 0.75-2.5 mm or 1-2 mm or approximately 1.5 mm. As a result, the upper portion 242 of the elongated cells can be a sized to provide adequate blood flow and/or access via a re-access device.

In some embodiments, outflow edges of the leaflets 84a and 84b, away from the commissure (and commissure tabs 84a and 84b, toward a center of the valve (toward the central longitudinal axis of the frame)) can be higher (closer to the outflow end 54) or lower than the outflow edges of the leaflets 84a and 84b at the commissure tabs 84a and 84b.

In some embodiments the outflow edges of the leaflets 84a and 84b, away from the commissure (and commissure tabs 84a and 84b, toward a center of the valve (toward the central longitudinal axis of the frame)) can also be offset from the outflow end 54 of the frame 200 by the axial distance 250.

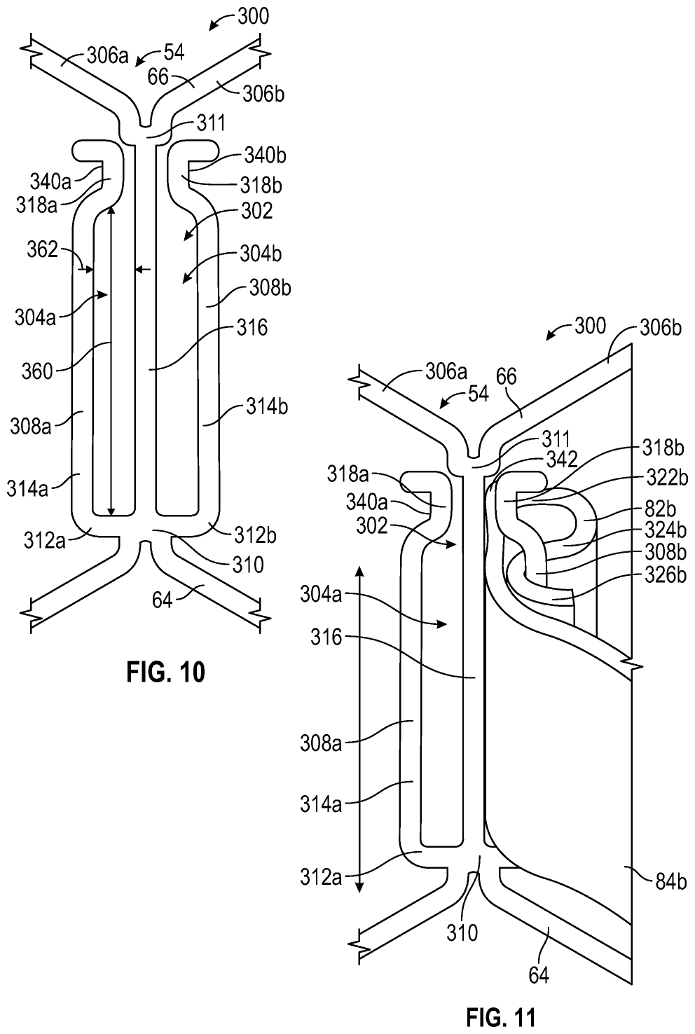

In yet another embodiment, as shown in FIGS. 10, 11, 13, and 14, a frame 300 having elongated cells at an outflow end 54 of the frame 300 can have a plurality of open commissure windows 302 (only one shown in FIGS. 10, 11, 13, and 14), split into two open window portions, the two open window portions formed between an axial strut (e.g., axially extending strut, similar to axially extending struts 70) and two axial strut arms disposed on either side of the axial strut. In some embodiments, each of the axial strut and two axial strut arms may be referred to as axially extending window struts (e.g., since they form the two open window portions of the open commissure window 302). The two open window portions are separated from one another via the axial strut and are each configured to receive a different commissure tab of a commissure therein.

More specifically, as shown in FIGS. 10 and 11, each open commissure window 302 can include a first open window portion 304a and a second open window portion 304b. The first open window portion 304a is formed between (and defined by) the axial strut 316 and a first axial strut arm 308a and the second open window portion 304b is formed between (and defined by) the axial strut 316 and a second axial strut arm 308b.

The first and second axial strut arms 308a and 308b can be integrally formed with the frame 300, extending from a lower elongated strut junction 310 of the frame 300, toward the outflow end 54 of the frame 300, in parallel to the axial strut 316. The lower elongated strut junction 310 can be a junction between two angled struts 64 of two adjacent elongated cells 306a and 306b. Further, the axial strut 316 extends between the lower elongated strut junction 310 and an upper elongated strut junction 311. The upper elongated strut junction 311 can be a junction between two angled struts 66 of the two adjacent elongated cells 306a and 306b.

Each of the first and second axial strut arms 308a and 308b are laterally offset (in opposite directions) from the axial strut 316. In some embodiments, the lateral direction may also be referred to as a circumferential direction (since the frame 300 is annular).

In some embodiments, each of the first and second axial strut arms 308a and 308b can include a lateral portion 312a and 312b, respectively, that extends between the lower elongated strut junction 310 and a respective axial portion 314a and 314b of the first and second axial strut arms 308a and 308b. The lateral portions 312a and 312b can be arranged substantially perpendicular to the axial portions 314a and 314b.

An upper end of each of the first and second axial strut arms 308a and 308b, the upper end arranged proximate to the outflow end 54 of the frame 300, can be free (e.g., not connected to another strut or portion of the frame 300). As a result, the first and second axial strut arms 308a and 308b can be individually bent or displaced away from the axial strut 316, in a lateral and/or radial direction, and then revert back to their axial orientation (e.g., approximately in parallel and radially aligned with the axial strut 316) when the bending or displacement force is no longer being applied thereto. As explained further below, the upper end of each of the first and second axial strut arms 308a and 308b can comprise a respective upper clamping portion 318a and 318b.

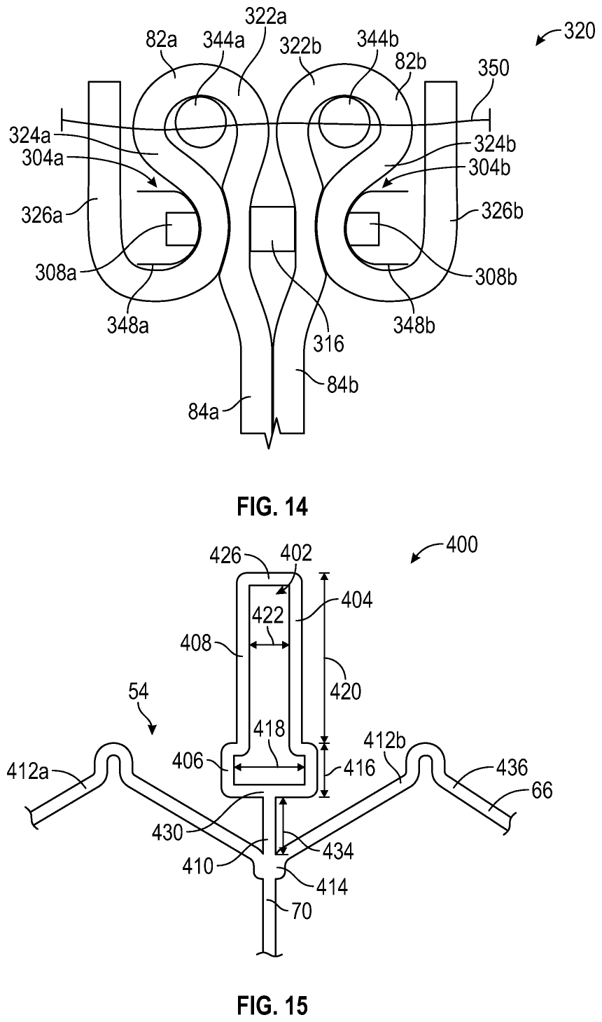

As shown in FIGS. 11, 13, and 14, commissure tabs 82a and 82b of adjacently arranged leaflets 84a and 84b, respectively, can extend through corresponding open window portions 304a and 304b of the open commissure window 302 to form a commissure 320 (complete commissure 320 shown in FIGS. 13 and 14).

In some embodiments, a resting (e.g., unbent or unflexed) width 362 of the open window portions 304a and 304b can be selected based on a thickness of the commissure tabs 82a and 82b (e.g., such that each open window portion is slightly narrower than or a same width as two times the commissure tab thickness).

For example, commissure tabs 82a and 82b of leaflets 84a and 84b can extend through open window portions 304a and 304b, respectively, for example, by flexing or bending the axial strut arms 308a and 308b laterally outward (away from the axial strut 316) and/or radially inward (toward the central longitudinal axis of the frame 300). This may ease the process of commissure tab insertion into the open window portions 304a and 304b.

As shown in FIGS. 11, 13, and 14, the second commissure tab 82b can extend radially outward from a body of the leaflet 84b, toward the frame 300, and through the second open window portion 304b. The second commissure tab 82b tab can then be folded over itself (e.g., folded over from its first tab portion 322b), to form a second tab portion 324b which extends radially inward through the same second open window portion 304b. The second tab portion 324b can be folded laterally outward from the axial strut 316 and over the corresponding, second axial strut arm 308b, thereby forming a third tab portion 326b.

As shown in FIGS. 11 and 13, in some embodiments, the third tab portion 326b can be disposed on the radial inner side of the frame 300 (e.g., radially inward-facing side relative to the central longitudinal axis of the frame). Similarly, as shown in FIG. 13, the first commissure tab 82a can extend radially outward from a body of the leaflet 84a, toward the frame 300, and through the first open window portion 304a. The first commissure tab 82a tab can then be folded over itself (e.g., folded over from its first tab portion 322a), to form a second tab portion 324a which extends radially inward through the same first open window portion 304a. The second tab portion 324a can be folded laterally outward from the axial strut 316 and over the corresponding, first axial strut arm 308a, thereby forming a third tab portion 326a. It should be noted that only the second commissure tab 82b is shown in FIG. 11 for ease of illustration, but the first commissure tab 82a can be folded as described above and shown in FIG. 13.

In alternate embodiments, as shown in FIG. 14, the third tab portions 326a and 326b can be further folded over a laterally outward-facing side of the respective axial strut arm 308a and 308b, thereby extending radially outward toward the radially outward-facing side of the frame 300.

As introduced above, in some embodiments, each of the first and second axial strut arms 308a and 308b includes a respective upper clamping portion 318a and 318b. For example, in some embodiments, as shown in FIGS. 10 and 11, a first upper clamping portion 318a bends from the axial portion 314a of the first axial strut arm 308a, laterally inward toward the axial strut 316, extends upward toward the outflow end 54 of the frame 300, and then bends laterally outward and away from the axial strut 316. As a result, a first indented portion 340a is formed in the first upper clamping portion 318a. Similarly, a second upper clamping portion 318b bends from the axial portion 314b of the second axial strut arm 308b, laterally inward toward the axial strut 316, extends upward toward the outflow end 54 of the frame 300, and then bends laterally outward and away from the axial strut 316. As a result, a second indented portion 340b is formed in the second upper clamping portion 318b. Together, the first and second indented portions 340a and 340b of the upper clamping portions 318a and 318b form a neck region at the upper (e.g., proximal) end of the commissure window 302, adjacent to the outflow end 54 of the frame 300.

In some embodiments, a fastener (e.g., a suture, band, tie, or the like) can be wrapped or looped around the first and second indented portions 340a and 340b of the upper clamping portions 318a and 318b, after the commissure tabs have been extended through the open window portions 304a and 304b, respectively. As a result, the upper ends of the first and second axial strut arms 308a and 308b are clamped together and to the axial strut 316, thereby preventing the commissure tabs from sliding axially out of the open window portions 304a and 304b.

In some embodiments, a distance (e.g., axial length) 360 between the lower elongated strut junction 310 and one of the first and second upper clamping portions 318a and 318b corresponds to a height (in the axial direction) of the commissure tabs 82a and 82b.

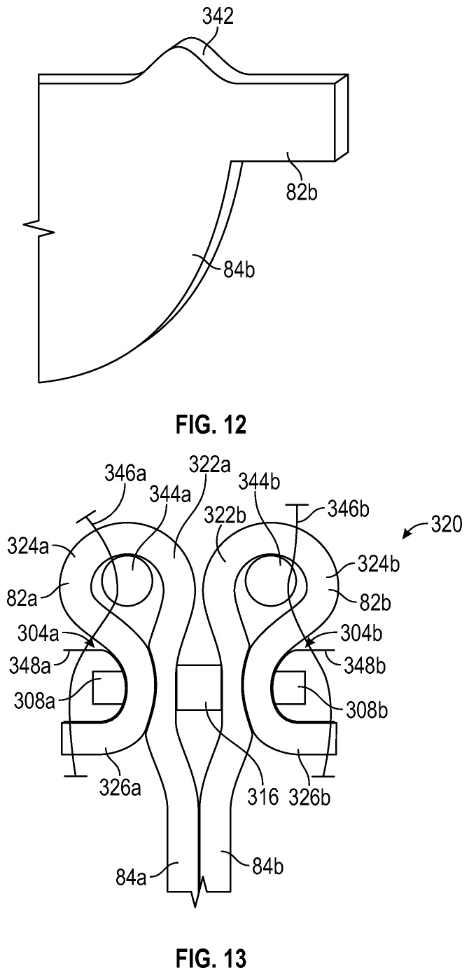

In some embodiments, as shown in FIG. 12, the leaflet 84b (and similarly, leaflet 84a) can include an axial extension 342, formed as a bump extending axially, in a proximal (upper) direction, at a region of transition between the leaflet 84b and the commissure tab 82b. As shown in FIG. 11, the axial extension 342 is aligned with the upper clamping portion 318b, such that the axial extension 342 is positioned between, in the lateral direction, the upper clamping portion 318b and the axial strut 316. Though only the second leaflet 84b is shown in FIG. 12, the first leaflet 84a can also include the axial extension 342, arranged between the upper clamping portion 318a and the axial strut 316 when inserted into the first open window portion 304a.

In this embodiment, when a suture or other fastener is looped and tightened around the upper clamping portions 318a and 318b, the axial extensions 342 of the leaflets 84a and 84b are pinched and clamped between the axial strut 316 and the respective upper clamping portion 318a and 318b. Thus, the axial extensions 342 may serve to relive stresses applied to the leaflets 84a and 84b during the transition between the systolic and diastolic phases (during operation of the prosthetic heart valve, in vivo).

FIGS. 13 and 14 show a sectional top view of two embodiments of the commissure 320 arranged within the commissure window 302 formed by the frame 300. In some embodiments, as shown in FIGS. 13 and 14, the leaflet first tab portions 322a and 322b can be folded over a respective reinforcing member 344a and 344b, forming the second tab portions 324a and 324b, respectively, extending therefrom toward the respective open window portions 304a and 304b. In some embodiments, the reinforcing members 344a and 344b can be referred to as wedge members.

In some embodiments, as shown in FIG. 13, sutures 346a and 346b can extend from the radially inward-facing side of the frame 300 to the radially outward-facing side of the frame, laterally outward of the respective axial strut arm 308a and 308b. For example, as shown in FIG. 13, each of the sutures 346a and 346b can extend through three layers of the respective commissure tab 82a and 82b, including the respective first tab portion 322a and 322b, second tab portion 324a and 324b, and third tab portion 326a and 326b. In some embodiments, the sutures 346a and 346b cab further extend through the respective reinforcing member 344a and 344b disposed between the first and second tab portions.

In some embodiments, fabric strips 348a and 348b can be disposed between the second tab portions 324a and 324b, the third tab portions 326a and 326b and the axial strut arms 308a and 308b, respectively. As such, the fabric strips 348a and 348b may reduce abrasion of the commissure tabs 82a and 82b against the axial strut arms 308a and 308b. In some embodiments, the sutures 346a and 346b can extend through flaps or end portions of the fabric strips 348a and 348b, respectively.

In some embodiments, fabric strips (such as fabric strips 348a and 348b) can be further disposed between the commissure tabs 82a and 82b and the axial strut 316 (not shown in FIG. 13).

In alternate embodiments, as shown in FIG. 14, the third tab portions 326a and 326b are further folded over a laterally outward-facing side of the respective axial strut arm 308a and 308b, thereby extending radially outward to (and, in some embodiments, outward of) the radially outward-facing side of the frame 300. In these embodiments, a single suture 350 can extend, on the outer side of the frame 300, in a lateral direction (opposed to the radial direction shown in FIG. 13), through all three layers of both commissure tabs 82a and 82b (e.g., through six leaflet tab layers in total).

In some embodiments, the suture 350 can further extend through the reinforcing members 344a and 344b.

In some embodiments, the commissure tabs 82a and 82b can be folded over the reinforcing members 344a and 344b to form partial pre-assemblies, so as to simplify their insertion and attachment into the commissure windows of the frame 300 disclosed above. For example, the commissures 320 can be assembled outside of the frame 300 by folding and securing the commissure tabs as explained above with reference to FIGS. 11, 13, and/or 14 and then the assembled commissures 320 can be inserted (e.g., slid) into the commissure window 302 via the openings at the upper end of the commissure window 302, formed by the free ends of the axial strut arms 308a and 308b (e.g., at the clamping portions 318a and 318b). Specifically, an assembled commissure 320 can be slid from above into the commissure window (e.g., the first folded commissure tab 82b into the first open window portion 304a via an opening between the axial strut 316 and the upper clamping portion 318a and the second folded commissure tab 82b into the second open window portion 304b via an opening between the axial strut 316 and the upper clamping portion 318b). In this way, the assembly of leaflets into the prosthetic heart valve may be simplified and made easier, thereby saving manufacturing time and effort.

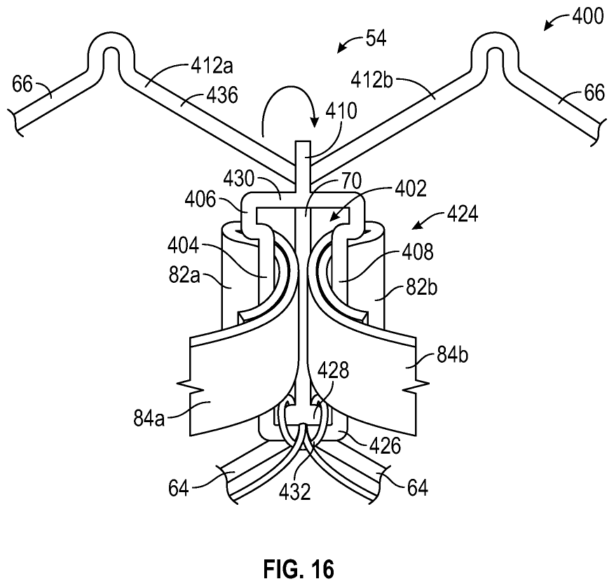

In another embodiment, as shown in FIGS. 15 and 16, a frame 400 having elongated cells at an outflow end 54 of the frame 400 can have a plurality of commissure windows 402 (only one shown in FIGS. 15 and 16), which are integral with a remainder of the frame. Each commissure window 402 can extend from an upper edge 436 (e.g., at the outflow end) of the frame 400 and be configured to bend from an extended configuration to a bent configuration where the commissure window 402 is overlapping an axially extending strut of the upper cells of the frame 400. The commissure window 402 can be a closed window formed by a strut frame 404 including a wider, first portion 406 that is connected to the outflow end 54 of the frame 400 via a bendable strut portion 410 and a narrower, second portion 408 that is configured to retain a commissure of a leaflet assembly therein. The bendable strut portion 410 can be configured to bend via manual force and retain its bent shape (e.g., plastically deformable).

More specifically, as shown in FIGS. 15 and 16, the frame 400 of the prosthetic heart valve can include an upper row of elongated cells (similar to frame 50 shown in FIGS. 2 and 3). FIGS. 15 and 16 are detail views of a portion of the frame 400, including two adjacent elongated cells of the upper row of cells, including a first elongated cell 412a and a second elongated cell 412b. The first and second elongated cells 412a and 412b share an axial strut 70 which extends between an upper elongated strut junction 414 (as shown in FIG. 15), which is a junction between lower ends (e.g., ends arranged inward of and farthest away from the outflow end 54) of two angled struts 66, and a lower strut junction 428 (shown in FIG. 16) which is a junction between upper end (e.g., ends arranged closer to the outflow end 54) of two angled struts 64.

As introduced above, the frame 400 can include a plurality of integrally-formed commissure windows (only one commissure window 402 shown in FIGS. 15 and 16) spaced apart around a circumference of the frame 400. Each commissure window 402 can extend from the upper (e.g., proximal) edge 436 of the frame (e.g., the edge arranged closest to the outflow end 54 and comprising angled struts 66) and is adapted to bend over the axial struts (e.g. axial strut 70) of the upper cells.

As shown in FIG. 15, the commissure window 402 is formed as a closed window (e.g., no openings around its perimeter), enclosed by the strut frame 404. The strut frame 404 can comprise a plurality of connected strut portions, that are continuous with one another, and that form the wider, first portion 406 and the narrower, second portion 408 of the strut frame 404. The second portion 408 is directly connected to the upper edge 436 of the frame 400, at one of the upper elongated strut junctions 414 via the bendable strut portion 410.

In some embodiments, the frame 404 of the commissure window 402 is formed by known manufacturing methods, such as laser cutting.

The bendable strut portion (also referred to as a bendable strut) 410 can comprise an axial strut (e.g., arranged parallel to the central longitudinal axis of the frame 400) that is configured to bend. In some embodiments, the bendable strut portion 410 is flexible and plastically deformable, to allow the commissure window 402 to translate from an upwardly-extended configuration (also referred to as a fully extended configuration, as shown in FIG. 15) to a fully bent or flipped configuration (as shown in FIG. 16). Thus, in some embodiments the bendable strut portion 410 can comprise a plastically deformable material, such as a plastically deformable metal or plastic.

In this way, the bendable strut portion 410 can be configured to allow bending of the commissure window 402, about the upper elongated strut junction 414 via the bendable strut portion 410, via manual force, and retain the bent orientation when the manual force (exceeding a set threshold) is no longer applied to the commissure window 402. As a result, spontaneous or unwanted displacement of the commissure window 402 from a selected orientation may be avoided.

As shown in FIG. 15, the first portion 406 has a first length 416 (in the axial direction) and a first width 418 (in the lateral or circumferential direction) and the second portion 408 has a second length 420 and a second width 422. The second length 420 is longer than the first length 416 and the first width 418 is wider than the second width 422. The lengths and widths of the first and second portions can be selected to accommodate commissure tabs of a commissure and arrange the leaflets within a lower portion of the upper cells, as explained further below.

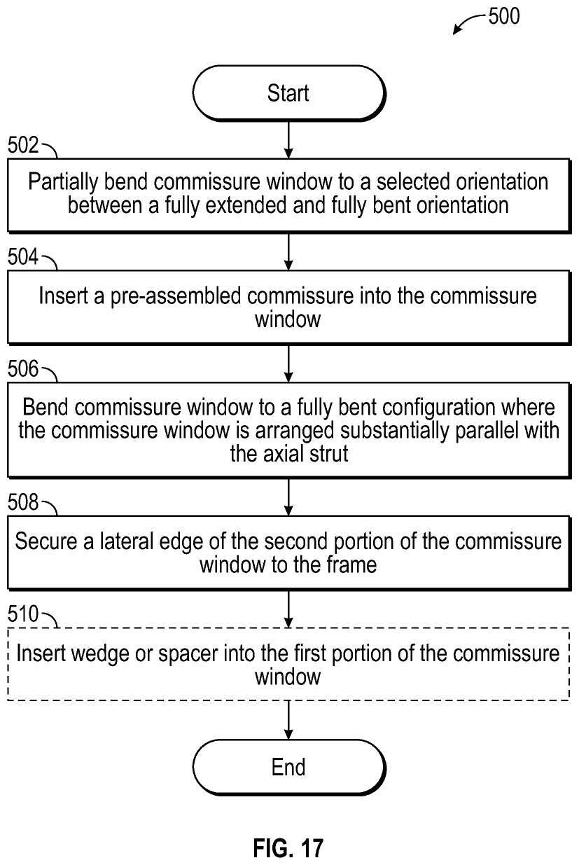

FIG. 17 shows a method 500 for assembling a commissure within the commissure window 402 and securing the commissure window containing the commissure to the frame 400. An example commissure 424 assembled within the commissure window 402 and secured to the frame 400 is shown in FIG. 16. The commissure 424 can comprise commissure tabs 82a and 82b of adjacently arranged leaflets 84a and 84b, respectively, as shown in FIG. 16.

Method 500 begins at 502 by at least partially bending the commissure window 402 to a selected orientation between the fully extended, initial orientation (e.g., as shown in FIG. 15) and the fully bent (e.g., flipped) orientation (as shown in FIG. 16). The fully extended orientation may be an orientation where the bendable strut portion 410 is not bent and extends substantially in the axial direction, outward and away from the upper elongated strut junction 414. In some embodiments, the method at 502 can include bending the commissure window 402 by 90°-135°, relative to the fully extended orientation. In other embodiments, the method at 502 can include bending the commissure window 402 by 80°-160°, relative to the fully extended orientation. In still other embodiments, the method at 500 can include bending the commissure window 402 by 75°-140°, relative to the fully extended orientation.

Method 500 continues to 504 to insert a pre-assembled commissure into the commissure window 502. In some embodiments each commissure can be pre-assembled outside the prosthetic heart valve frame 400. For example, in some embodiments, the pre-assembled commissure can comprise two commissure tabs of two adjacent leaflets (e.g., leaflets that will be adjacently arranged within the frame 400) folded into a desired configuration and secured together via one or more reinforcing (e.g., wedge) members and/or fasteners (e.g., sutures). The desired folded configuration of the commissure tabs can comprise one or more overlapping layers of the commissure tabs which may result in a more secure fit within the commissure window and reduce the likelihood of the commissure being pulled through and out of the commissure window after installation within the frame (e.g., during valve operation in vivo).

In some embodiments, insertion of the commissure 424 into the commissure window 402 can be facilitated by the wider, first portion 406 of the commissure window 402. For example, the commissure 424 can be inserted first into the first portion 406 and then slid and extended into the narrower and elongated second portion 408. In this way, the wider, first width 418 can be selected to allow for easy insertion of the commissure tabs of the commissure 424 therein and the narrower, second width 422 can be selected to securely hold the commissure tabs of the commissure 424 therein.

For example, the second length 420 and second width 422 of the second portion 408 can be selected to accommodate the commissure 424 therein (e.g., having a length and width configured to tightly retain the commissure tabs of the commissure therein). Additionally, in some examples, the first length 416 and first width 418 of the first portion 406 can be selected to allow insertion (e.g., with reduced effort) of the commissure into the commissure window 402, during the assembly process.

Further, in some embodiments, a length 434 of the bendable strut portion 410 and the dimensions (e.g., lengths and widths described above) of the first and second portions of the strut frame 404 of the commissure window 402 can be selected so as to position the second portion 408 of the commissure window 402 closer to the lower portion of the elongated cells (e.g., elongated cells 412a and 412b), thereby maintaining the first portion 408 (containing the commissure 424) spaced away from the upper edge 436 of the elongated cells. As a result, the leaflets can be positioned in a lower profile configuration, within a lower portion of the elongated cells, while leaving an upper portion (or region) of the upper, elongated cells unblocked. As discussed herein, this configuration may allow for re-access and/or ensuring adequate blood flow through the upper, elongated cells, following valve-in-valve procedures.

In some embodiments, the first length 416, minus two times a thickness of the frame 404, can be approximately 2 mm (e.g., an interior length, or length of the open space, of the first portion 406), the first width 418 can be approximately 3 mm, and the second width 422 can be approximately 0.8 mm. In some embodiments, the first length 416, minus two times the thickness of the frame 404, can be in a range of 1.5-2.5 mm, the first width 418 can be in a range of 2.5-3.5 mm, and the second width 422 can be in a range of 0.6-1.0 mm. It should be noted that the above embodiments of the strut frame dimensions are exemplary and not meant to be limiting.

Returning to FIG. 17, method 500 can proceed from 504 to 506 to further bend the commissure window 402 (with the commissure arranged therein) to the fully bent configuration, where the commissure window 402 is arranged substantially parallel with the axial strut 70 (as shown in FIG. 16). In this fully bent configuration, the commissure window 402 can be arranged radially inward, relative to the central longitudinal axis of the frame 400, the axial strut 70. In some embodiments, the commissure window 402 can be aligned, in the lateral (or circumferential) direction with a central, longitudinal axis of the commissure window 402. For example, as shown in FIG. 16, when in its fully bent configuration, the commissure window 402 can be arranged directly in front, relative to the radial direction, the axial strut 70.

Method 500 then proceeds to 508 to secure (e.g., attach) a lateral edge 426 (e.g., may be referred to as a lower edge in the fully bent configuration) of the second portion 408 of the commissure window 402 to the frame 400. The lateral edge 426 is arranged opposite to a lateral edge 430 of the first portion 406. In some embodiments securing the lateral edge 426 to the frame 400 includes securing (e.g., via one or more fasteners, such as one or more sutures 432, as shown in FIG. 16) the lateral edge 426 to the lower strut junction 428 and/or to strut portions (e.g., angled struts 64) extending from the lower strut junction 428.

In some embodiments, method 500 can further include, at 510, inserting a wedge or spacer into the first portion 406, after the commissure 424 is arranged within the second portion 408, as described above. In some embodiments, the wedge or spacer can be fastened (e.g., sutured) above the commissure tabs 82a and 82b. As such, the commissure tabs 82a and 82b may be wedged tightly within the second portion 408 of the frame 404 of the commissure window 402, thereby preventing radial displacement of the commissure 424 within the commissure window 402. The wedge or spacer can include a polymeric or fabric material, in some embodiments.

In this way, the commissure window and frame configuration discussed above with reference to FIGS. 15-17 can be manufactured and assembled more easily since the commissures of the leaflets can be pre-assembled outside the frame and more easily inserted through the wider, first portion of the commissure windows. Further, the ability to orient the commissure windows to any desirable angle (relative to the frame) during the assembly process, may further simplify the process of commissure attachment to the frame.

Additionally, by reducing the number of separately attachable components, by providing commissure windows which are integrally formed with the frame, manufacturing costs may be further reduced, and the assembly process may be less time-consuming. Further, integrally formed commissure windows may prevent undesirable relative movement between otherwise attachable components, which may reduce or eliminate fretting corrosion of contacting components.

The frame embodiments disclosed above with reference to FIGS. 5-17 provide prosthetic heart valve frames with integrally-formed commissure windows, for frames having an upper row of cells (e.g., arranged at an outflow end of the frame) that are elongated in the axial direction relative to remaining rows of cells of the frame (e.g., lower cells arranged closer to an inflow end of the frame). As discussed above, these integral commissure windows are configured to maintain the leaflet assembly of the valve at a lower position within the upper row of cells, thereby creating a void space for increased blood flow and/or passage of re-access device through the upper row of cells.

In alternate embodiments, a frame of a prosthetic heart valve, such as frame 50 shown in FIGS. 2 and 3, can be configured to receive a commissure support element. The commissure support element can be separate from (e.g., not integrated with) the frame and be configured to be coupled (e.g., attached) to a portion of the frame. Further, the commissure support element can be configured to retain a commissure within an open window of the commissure support element.

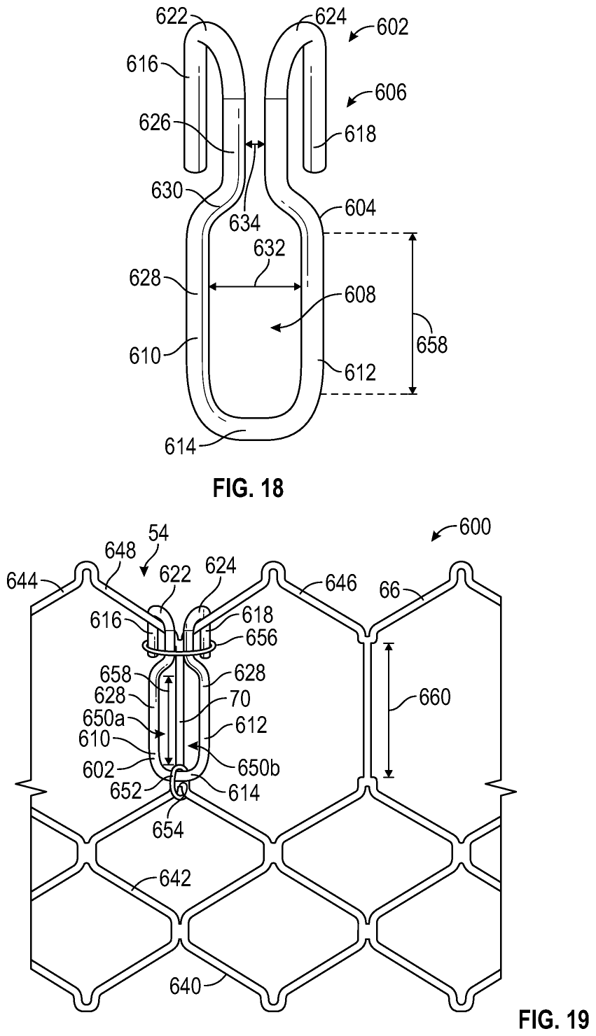

In one embodiment, as shown in FIGS. 18-21, a commissure support element 602 is provided that is attachable to a frame 600 of a prosthetic heart valve. In particular, the commissure support element 602 can be configured to attach to cells of an upper row of elongated cells of the frame 600 (e.g., cells forming elongated openings 76 in frame 50 of FIGS. 2 and 3). The commissure support element 602 can include a coupling portion adapted to couple to an upper edge of the upper row of cells, at an interface between two adjacent cells of the upper row of cells, and an open, leaflet-receiving window adapted to receive a commissure therein. In some embodiments, pre-assembled commissures, assembled outside the frame 600, can be slid into the open, leaflet-receiving window and secured to the commissure support element 602 and then the commissure support element 602 can be attached to the frame, thereby simplifying the assembly and manufacturing process of the prosthetic heart valve. Additionally, the commissure support element 602 can be further configured to arrange the commissure within a lower region of the upper row of cells, thereby leaving an upper region of the upper row of cells unblocked by the leaflets.

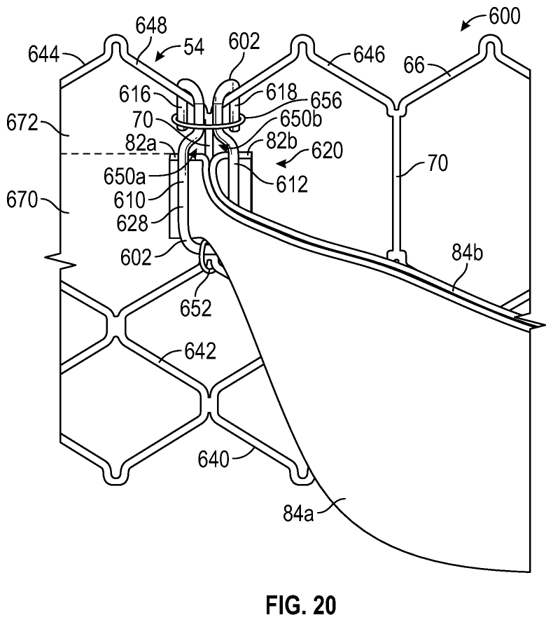

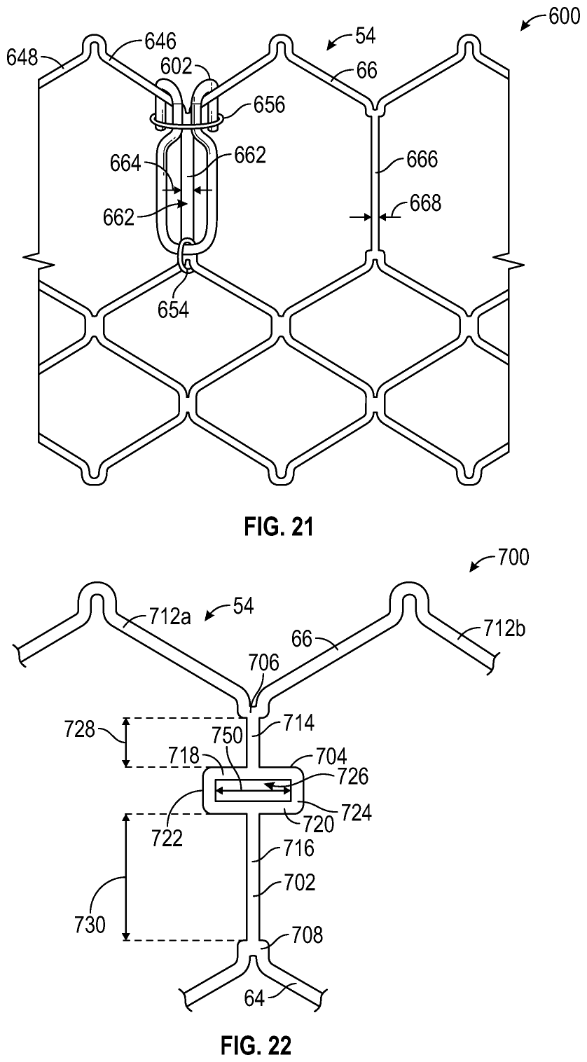

The commissure support element 602 is shown by itself in FIG. 18 and attached to the frame 600 in FIGS. 19-21. The commissure support element 602 can be configured as a unitary wire-form body 604 including a coupling portion 606 and a leaflet-receiving window 608 defined by axially extending first and second members (e.g., first and second axial members) 610 and 612 and a connecting member 614 (which extends substantially in the lateral direction, between the first and second axial members 610 and 612). The commissure support element 602 is shown with a commissure 620 arranged within the leaflet-receiving window 608 in FIG. 20 and without the commissure in FIGS. 19 and 21 (for better illustration of how the commissure support element 602 attaches to the frame 600).

In some embodiments, the wire-form body 604 can be formed by a known manufacturing process, such as tube-cutting, 3D printing, or another suitable process.

In some embodiments, as shown in FIG. 18, each of the first and second axial members 610 and 612 includes an upper axial portion 626 and a lower axial portion 628. For example, each lower axial portion 628 extends between the connecting member 614 and a bend 630 between the lower axial portion 628 and the upper axial portion 626. Each upper axial portion 626 extends between the bend 630 and a corresponding curved portion or member 622 or 624 (as described further below). As shown in FIG. 18, a width 632 between the lower axial portions 628 of each of the first and second axial members 610 and 612 is wider than a width 634 between the upper axial portions 626 of each of the first and second axial members 610 and 612. The width 632 is a width of the leaflet-receiving window 608 (also referred to as a commissure window).

The coupling portion 606 can comprise a pair of coupling members 616 and 618 that are radially offset from the first and second axial members 610 and 612. The coupling member 616 can be coupled to the first axial member 610 by a curved portion or member 622, and the coupling member 618 can be coupled to the second axial member 612 by a curved portion or member 624. In certain embodiments, the curved members 622 and 624 can curve by 180° such that the first and second axial members 610 and 612 extend parallel or substantially parallel to the coupling members 616 and 618, for example toward the inflow end of a prosthetic heart valve of which the frame 600 is part, although other configurations are possible.

In some embodiments, as shown in FIG. 18, the angular or circumferential spacing between the coupling members 616 and 618 can be greater than the angular spacing between the upper axial portions 626 of the first and second axial members 610 and 612. Accordingly, the curved members 622 and 624 can be angled toward one another.

As introduced above, the members 610, 612, and 614 can at least partially define the leaflet-receiving window 608, which can be open at the top (e.g., open at the end of the commissure support element 602 including the curved members 622 and 624). As a result, in some embodiments, an assembled commissure can be slid into the leaflet-receiving window 608 through a space between the curved members 622 and 624 and through a space between the upper axial portions 626 (e.g., from above), as described further below.

FIGS. 19-21 show a portion of the exemplary frame 600, which may be similar to frame 50 of FIGS. 2 and 3, as described above. For example, the frame 600 can include a plurality of rows of cells, where first and second rows of cells, arranged closer to a distal end 640 of the frame 600 (which can be an inflow end of the frame), include cells 642 defined by four strut portions extending between four junctions or apices (illustrated in FIGS. 19-21 as approximately diamond-shaped cells, but the cells may assume other shapes, for example if the strut portions are curved). An upper row of cells, arranged closer to a proximal end 644 of the frame (which can be the outflow end 54 of the frame 600), includes elongated cells 646, defined by six strut portions, two of which are elongated strut portions, referred to herein as axial struts 70, extending in an axial direction on both sides of the cell 646.

The frame 600 and other frames disclosed herein can include any other number of cell rows with cells 642, as long as the upper (i.e., proximal) row includes the elongated cells 646. As introduced above, in some embodiments, this configuration may be utilized for smaller diameter valves, where the elongated cells 646 provide a larger cell opening, through which coronary re-access procedures can be performed.

As shown in FIGS. 19 and 20, the commissure support element 602 can be coupled to the elongated cells 646. More specifically, the commissure support element 602 can be coupled to angled struts 66, forming an upper edge 648, of two adjacent elongated cells 646. For example, as shown in FIGS. 19 and 20, the coupling members 616 and 618 hook around the angled struts 66 of the two adjacent elongated cells (e.g., coupling member 616 over an angled strut 66 of a first elongated cell of the two adjacent elongated cells 646 and coupling member 618 over an angled strut 66 of a second elongated cell of the two adjacent elongated cells 646).

In this coupling configuration, the coupling members 616 and 618 of the commissure support element 602 are positioned radially outward of the frame 600 (relative to the central longitudinal axis of the annular frame 600) and the first and second axial members 610 and 612 are positioned radially inward of the frame 600. Additionally, in this coupling configuration, the leaflet-receiving window 608 is positioned substantially radially inward of the elongated, axial strut 70 (which forms a part, or common axial side, of each of the two adjacent elongated cells to which the commissure support element 602 is coupled). As a result, the axial strut 70 divides the leaflet-receiving window 608 of the commissure support element 602 into two open window portions: a first window portion 650a defined between the axial strut 70 and the lower axial portion 628 of the first axial member 610 and a second window portion 650b defined between the axial strut 70 and the lower axial portion 628 of the second axial member 612.

As shown in FIG. 20, the commissure tabs 82a and 82b of the commissure 620 can be inserted into the leaflet-receiving window 608 (for example, slid from above and downward into the leaflet-receiving window 608, prior to attaching the commissure support element 602 to the frame 600) such that a first commissure tab 82a extends through the first window portion 650a and a second commissure tab 82b extends through the second window portion 650b. For example, in some embodiments, the first and second commissure tabs 82a and 82b can be coupled together to form a commissure 620. The formed commissure 620 may then be slid into the leaflet-receiving window 608 of the commissure support element 602, while the commissure support element 602 is detached from the frame 600 (as shown in FIG. 18). The commissure support element 602, containing the commissure 620, can then be coupled to the frame, as shown in FIG. 20.

In some embodiments, after attaching the commissure support element 602 to the frame 600, a first fastener (e.g., suture) 652 can be used to attach the connecting member 614 to the frame 600. In some embodiments, the connecting member 614 can be coupled to a lower strut junction 654 of the frame 600 (as shown in FIG. 19).

In some embodiments, a second fastener (e.g., one or more sutures) 656 can be used to secure the upper axial portions 626 of the first and second axial members 610 and 612 and/or the coupling members 616 and 618 to the frame 600 (e.g., to the angled struts 66 forming the upper edge 648 of the two adjacent elongated cells 646).

An axial length 658 of the lower axial portions 628 can be configured so that the first and second window portions 650a and 650b, which are configured to receive the commissure tabs 82a and 82b, respectively, of the commissure 620, are shorter than the length 660 of the axial strut 70. As a result, the commissure 60 is positioned within a lower region 670 of the elongated cells 646 so that the leaflets 84a and 84b do not block at least an upper region 672 of the elongated cells' opening. The unblocked, upper region of the elongated cells 646 can be large enough to allow for sufficient blood flow to the coronary ostia after valve implantation, as well as allow coronary re-access devices to pass therethrough.

In alternate embodiments, the commissure support element can be configured as a relatively planar member, wherein the curved members 622 and 624 offset the coupling members 616 and 618 from the first and second axial members 610 and 612 only in the lateral direction (e.g., not radially outward). In such embodiments, all portions of the commissure support element, including the coupling members 616 and 618, reside along an inner surface of the frame 600 (e.g., in a same plane) when the commissure support element is coupled to the frame. In these embodiments, the commissure tab attachment and member suturing around the frame may be executed in a similar manner as described above. Such a configuration of the commissure support element may reduce the crimp profile of the valve, without having coupling members 616 and 618 extending outward of the frame 600.

During operation of the prosthetic heart valve, having frame 600, in vivo, the leaflets of the valve are movable from an open state during the systolic phase to a closed state during the diastolic phase. The constant cyclical movement of the leaflets may exert forces on the axial struts 70 that may cause material fatigue and degradation over time. Thus, in some embodiments, as shown in FIG. 21, the axial struts 662 that are configured to be coupled with the commissure support elements 602 can have a greater thickness 664 than a thickness 668 of the axial struts 666 that are not configured to be coupled with the commissure support elements 602. As such, the thicker axial struts 666 may be better adapted to withstand the increased stresses.

In another embodiment, as shown in FIGS. 22-24, a frame 700 having elongated cells at an outflow end 54 of the frame 700 (e.g., similar to frame 50 of FIGS. 2 and 3) can have axial struts 702 including a strut window 704. For example, adjacent elongated cells share an axial strut that extends between an upper elongated strut junction 706, which is a junction between lower ends (e.g., ends arranged inward of and farthest away from the outflow end 54) of two angled struts 66, and a lower elongated strut junction 708, which is a junction between upper ends (e.g., ends arranged closer to the outflow end 54) of two angled struts 64. The axial struts of the upper rows of cells can either be straight axial struts 70 (as shown in FIGS. 2 and 3) or window axial struts 702 (as shown in the detail views of FIGS. 22-24) that include the strut window 704 which is configured to receive a commissure support element 710, the commissure support element 710 configured to receive a commissure. For example, the frame 700 can include three window axial struts 702 (spaced apart from one another around a circumference of the annular frame 700) while the remaining axial struts forming the upper row of cells are the straight axial struts 70 (e.g., not having the strut window 704). The number of axial struts 702 having the strut window 704 can correspond to the number of commissures of the leaflet assembly of the prosthetic heart valve. In alternate embodiments, the frame 700 may include more or less than three axial struts 702.

As described further below, the commissure support element 710 can include a coupling portion adapted to couple to the strut window 704 and an open, leaflet-receiving window 734 adapted to receive a commissure therein. In some embodiments, pre-assembled commissures, assembled outside the frame 700, can be slid into the open, leaflet-receiving window 734 and secured to the commissure support element 710 and then the commissure support element 710 can be attached to the strut window 704 of the axial strut 702, thereby simplifying the assembly and manufacturing process of the prosthetic heart valve. Additionally, the strut window 704 can be positioned at a selected position along a length of the axial strut 702 in order to arrange the commissure within a lower region of the elongated cells of the upper row of cells, thereby leaving an upper region of the elongated cells of the upper row of cells unblocked by the leaflets.

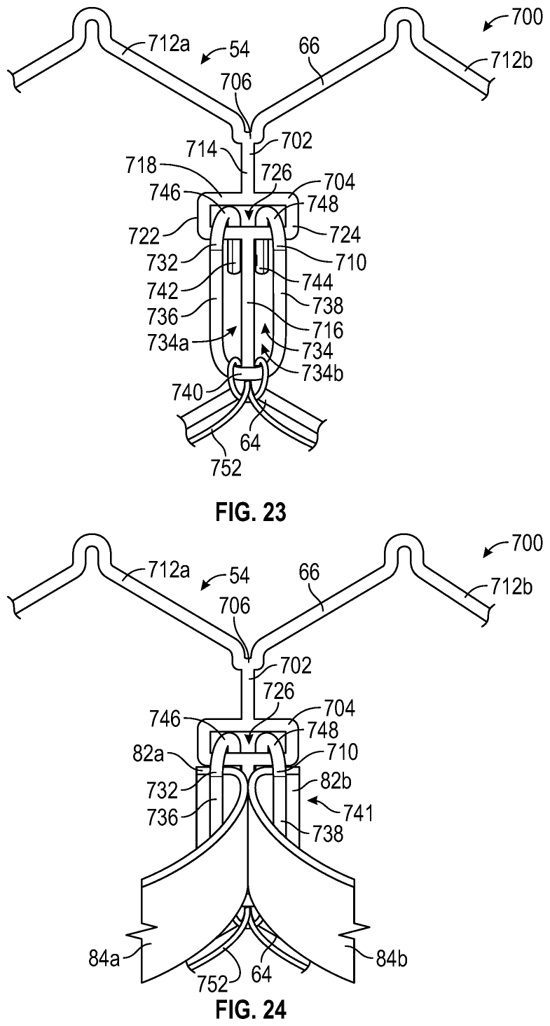

FIGS. 22-24 show a detail portion of an exemplary annular frame 700 including one axial strut 702, including the strut window 704, arranged between and forming an axial side of each of a first elongated cell 712a of the upper row of cells and a second elongated cell 712b of the upper row of cells. The first and second elongated cells 712a and 712b are adjacently arranged within the upper row of cells of the frame 700. As discussed above, a remainder of the frame 700 may be similar to frame 50 of FIGS. 2 and 3 and/or the other frames described herein. While FIG. 22 shows the bare frame 700, FIG. 23 shows the commissure support element 710 attached to the strut window 704 of the frame 700, and FIG. 24 shows the commissure support element 710, with a commissure coupled therein, attached to the strut window 704.

As shown in FIG. 22, the axial strut 702 includes an upper (e.g., proximal) strut portion 714, a lower (e.g., distal) strut portion 716, and the strut window 704 arranged between the upper strut portion 714 and the lower strut portion 716. For example, the upper strut portion 714 extends between, in the axial direction, the upper elongated strut junction 706 and a first lateral strut portion 718 of the strut window 704 and the lower strut portion 716 extends between, in the axial direction, a second lateral strut portion 720 of the strut window 704 and the lower elongated strut junction 708.

The upper strut portion 714 has a first length 728 and the lower strut portion 716 has a second length 730. In some embodiments, as shown in FIG. 22, the first length 728 is shorter than the second length 730. As described further below, the first length 728 and the second length 730 can be selected so that the strut window 704 is spaced away from the upper elongated strut junction 706, thereby positioning the leaflet commissures within a lower region of the first and second elongated cells 712a and 712b.

Together, the first lateral strut portion 718, the second lateral strut portion 720, a first axial strut portion 722, and a second axial strut portion 724 of the strut window 704 form the closed strut window 704 and define a central opening 726 of the strut window 704. In this way, as shown in FIG. 22, the strut window 704 can be a closed, rectangular frame having the central opening 726. However, in alternate embodiments, the strut window 704 can have a different shape, such as oblong, circular, square, or the like.

The strut window 704 is configured to receive the commissure support element 710, within its central opening 726 (as shown in FIGS. 23 and 24). As show in FIG. 23, the commissure support element 710 can be configured as a unitary wire-form body 732 including a coupling portion configured to couple with the strut window 704 and a leaflet-receiving window 734 defined by axially extending first and second members (e.g., first and second axial members) 736 and 738 and a connecting member 740 (which extends substantially in the lateral direction, between the first and second axial members 736 and 738). The commissure support element 710 is shown with a commissure 741 arranged within the leaflet-receiving window 734 in FIG. 24 and without the commissure in FIG. 23 (for better illustration of how the commissure support element 710 attaches to the frame 700).

In some embodiments, the wire-form body 732 can be formed by a known manufacturing process, such as tube-cutting, 3D printing, or another suitable process.

In some embodiments, as shown in FIGS. 23 and 24, the coupling portion can comprise a pair of coupling members 742 and 744 that are radially offset from the first and second axial members 736 and 738. The coupling member 742 can be coupled to the first axial member 736 by a curved portion or member 746, and the coupling member 744 can be coupled to the second axial member 738 by a curved portion or member 748. In certain embodiments, the coupling members 742 and 744 can curve by 180° such that the first and second axial members 736 and 738 extend parallel or substantially parallel to the coupling members 742 and 744, for example in a direction of the central longitudinal axis of the frame 700, although other configurations are possible.

In some embodiments, as shown in FIGS. 23 and 24, the angular or circumferential spacing between the coupling members 742 and 744 can be smaller than the angular spacing between the first and second axial members 736 and 738.

As introduced above, the members 736, 738, and 740 can at least partially define the leaflet-receiving window 734, which can be open at the top (e.g., the end arranged closest to the outflow end 54 of the frame 700 when the commissure support element 710 is coupled to the strut window 704). As a result, in some embodiments, an assembled commissure (e.g., commissure 741) can be slid into the leaflet-receiving window 608 from above, as described further below.

As shown in FIGS. 23 and 24, the commissure support element 710 can be coupled to the strut window 704 by sliding the coupling member 742 and 744 through the central opening 726 such that the curved members 746 and 748 are placed over the second lateral strut portion 720 (which may form a lower edge) of the strut window 704.

When the commissure support element 710 is coupled to the strut window 704, as shown in FIGS. 23 and 24, the first and second axial members 736 and 738 are arranged radially inward of the frame 700, relative to the central longitudinal axis of the annular frame 700, and the coupling members 742 and 744 are arranged radially outward of the frame 700.

The central opening 726 of the strut window 704 can be sized (e.g., dimensioned) to accept the curved members 746 and 748 of the commissure support element 710. For example, in some embodiments, a width 750 (arranged in the lateral or circumferential direction) of the central opening 726 can be slightly smaller than the free-state distance between (in the lateral direction) outer edges of the curved members 746 and 748. As a result, a user may slightly move (e.g., via pinching) the curved members 746 and 748 toward each other during insertion into the strut window 704. In such embodiments, once positioned in the central opening 726, the curved members 746 and 748 can be laterally biased against the inner edges of the first and second axial portions 722 and 724 of the strut window 704. As a result, the commissure support element 710 may be more firmly retained within the strut window 704

As shown in FIG. 23, the leaflet-receiving window 734 of the commissure support element 710 can be arranged directly, radially inward of (e.g., covering) the lower strut portion 716, such that the lower strut portion 716 divides the leaflet-receiving window 734 into two window portions: a first window portion 734a defined between the lower strut portion 716 and the first axial member 736 and a second window portion 734b defined between the lower strut portion 716 and the second axial member 738.

As shown in FIG. 24, commissure tabs 82a and 82b can extend through corresponding first and second window portions 734a and 734b, respectively. In some embodiments, the commissure 741, including the commissure tabs 82a and 82b, is at least partially pre-assembled and inserted into the leaflet-receiving window 734 of the commissure support element 710, prior to coupling the commissure support element 710 to the frame 700.

In some embodiments, a first fastener (e.g., suture) 752 can be used to secure the connecting member 740 of the commissure support element 710 to the frame 700. For example, as shown in FIG. 23, the first fastener 752 can secure the connecting member 740 to the lower elongated strut junction 708.

In some embodiments, additional sutures (not shown) can be used to secure the first and second axial members 736 and 738 together and/or to secure the commissure support element 710 to the strut window 704.

In some embodiments, a wedge or spacer member (not shown) can be inserted between the curved members 746 and 748 and the first lateral strut portion 718 of the strut window 40, so as to reduce or prevent undesirable axial movement of the commissure support element within the central opening 726.

In some embodiments, the first and second axial members 736 and 738 of the commissure support element 710 are provided with a tapering cross-sectional geometry in the axial direction, such that their thickness increases toward their upper ends (e.g., the ends connected to the curved members 746 and 748). Such a configuration may assist in reducing the stress applied to the leaflets during diastole.

In this way, the frame 700 provides axial support to the commissure support element 710, since both the upper and lower portions of the commissure support element 710 are secured to the frame 700. Moreover, the disclosed strut window 704 may further restrict undesirable axial displacement of the commissure support element 710.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A method of assembling a prosthetic heart valve including an annular frame and a leaflet assembly, comprising: inserting a commissure, pre-assembled outside the annular frame, into a commissure window of the frame via an opening in an upper or lower portion of the commissure window, wherein the commissure comprises a pair of secured together commissure tabs of adjacently arranged leaflets of the leaflet assembly, wherein each commissure window is formed by one or more axially extending struts of two adjacent cells in an upper row of cells of the frame, the upper row of cells arranged at an outflow end of the frame and having an elongated length, in an axial direction relative to a central longitudinal axis of the frame, relative to cells in remaining rows of cells of the frame; and securing the commissure within a commissure-receiving portion of the commissure window via one or more fasteners such that the commissure is arranged within a lower portion of the upper rows of cells and an open area that is unblocked from leaflets is formed within an upper portion of the upper row of cells, the upper portion arranged closer to the outflow end than the lower portion.

Example 2. The method of any example herein, particularly example 1, wherein the commissure window is formed by two axially extending window struts that are spaced apart from one another in a circumferential direction, the circumferential direction relative to a circumference of the annular frame, a first of the two axially extending window struts forming an axial side of a first cell of the upper row of cells and a second of the two axially extending window struts forming an axial side of an adjacent, second cell of the upper row of cells, wherein the commissure window comprises a closed, lower region configured to receive commissure tabs of the commissure and an open, upper region arranged at the outflow end of the frame, and wherein securing the commissure with the commissure-receiving portion of the commissure window includes arranging the commissure within the lower region of the commissure window and securing a window fastener into the upper region of the commissure window via the one or more fasteners.

Example 3. The method of any example herein, particularly example 2, wherein securing the window fastener into the upper region includes first arranging a middle portion of the window fastener within the upper region of the commissure window such that a relatively constant distance is maintained between the two axially extending window struts and then looping one or more sutures around end portions of the window fastener, the end portions positioned radially inward and radially outward of the frame, the middle portion extending between the end portions, to secure the window fastener to the frame.

Example 4. The method of any example herein, particularly example 1, wherein the commissure window is formed by two axially extending window struts that are spaced apart from one another in a circumferential direction, the circumferential direction relative to a circumference of the annular frame, at their lower ends and along their length, the lower ends of the two axially extending window struts each connected to a corresponding lower angled strut of a corresponding one of the two adjacent cells, wherein the two axially extending window struts are connected together and to an upper axial strut, at their upper ends, via an upper edge of the commissure window that extends in the circumferential direction, wherein the upper axial strut is connected at its upper end to an upper elongated strut junction forming a junction between upper, angled struts of the two adjacent cells, and wherein the sliding the commissure through the opening includes sliding the commissure through the opening formed in the lower portion of the commissure window, between the spaced apart lower ends of the two axially extending window struts.

Example 5. The method of any example herein, particularly example 4, wherein the securing the commissure within the commissure-receiving portion of the commissure window includes wrapping and tightening one or more fasteners around lower clamping portions of the two axially extending window struts, the lower clamping portions forming an open, neck region at the lower ends of the two axially extending window struts, so that the lower ends of the two axially extending window struts are clamped together.

Example 6. The method of any example herein, particularly example 1, wherein the commissure window is divided into a first open window portion and a second open window portion, each of the first and second open window portions configured to receive a different one of the commissure tabs of the commissure therein, wherein the first open window portion is formed between a central, axially extending strut that extends between upper and lower angled struts of the two adjacent cells and a first axial strut arm connected to and laterally offset from, in a circumferential direction relative to a circumference of the annular frame, the axially extending strut, at a lower end of the first axial strut arm, wherein the second open window portion is formed between the axially extending strut and a second axial strut arm connected to a laterally offset from the axially extending strut, at a lower end of the second axial strut arm, and wherein inserting the commissure into the commissure window via the opening includes inserting a first commissure tab of the commissure into the first open window portion via an opening formed between a free, upper end of the first axial strut arm and the axially extending strut and inserting a second commissure tab of the commissure into the second open window portion via an opening formed between a free, upper end of the second axial strut arm and the axially extending strut.

Example 7. The method of any example herein, particularly example 6, wherein securing the commissure within the commissure-receiving portion of the commissure window includes tightening a fastener around the free, upper ends of the first and second axial strut arms to clamp the first and second axial strut arms together and to the axially extending strut.

Example 8. The method of any example herein, particularly any one of examples 1-7, wherein the annular frame is radially expandable to a diameter in a range of 19-21 mm.

Example 9. A prosthetic heart valve, comprising: an annular frame, comprising: a plurality of interconnected struts defining a plurality of rows of cells, the plurality of rows arranged between an outflow end and an inflow end of the frame and including an upper row of cells arranged at the outflow end and a lower row of cells arranged at the inflow end, wherein cells of the upper row of cells are elongated, in an axial direction relative to a central longitudinal axis of the frame, relative to cells of remaining rows of cells of the plurality of rows of cells, including the lower row of cells; and a plurality of commissure windows formed between axially extending window struts of the frame, each commissure window arranged between axially extending window struts of two adjacent elongated cells of the upper row of open cells, each commissure window having a commissure-receiving portion that is spaced away from an upper end of the two adjacent elongated cells, the upper end arranged at the outflow end of the frame, and an open end; and a leaflet assembly comprising a plurality of leaflets, each leaflet comprising opposing commissure tabs on opposite sides of the leaflet, each commissure tab being paired with an adjacent commissure tab of an adjacent leaflet to form a commissure, wherein each commissure is arranged within the commissure-receiving portion of a respective commissure window, and wherein the open end of the commissure window is configured to receive the commissure therethrough.

Example 10. The prosthetic heart valve of any example herein, particularly example 9, wherein the open end of each commissure window is formed by upper or lower ends of the axially extending window struts that are spaced apart from one another, in a circumferential direction, and not directly connected to one another, the circumferential direction relative to a circumference of the frame and arranged tangent to the axial direction.

Example 11. The prosthetic heart valve of any example herein, particularly any one of examples 9-10, wherein each commissure window is formed between a first axially extending window strut of a first elongated cell of the two adjacent elongated cells and a second axially extending window strut of a second elongated cell of the two adjacent elongated cells, the first and second axially extending window struts spaced apart from one another at their upper ends and along their lengths and connected together at their lower ends by a lateral window strut portion, wherein each of the first and second axially extending window struts comprise a lower window strut portion and an upper window strut portion, and wherein the commissure-receiving portion of the commissure is formed in a space between the lower window strut portion of the first axially extending window strut and the lower window strut portion of the second axially extending window strut.

Example 12. The prosthetic heart valve of any example herein, particularly example 9 or example 10, wherein each commissure window is formed between a first axially extending window strut and a second axially extending window strut that are spaced apart from one another in the circumferential direction at their lower ends and along their length, the lower end of each of the first and second axially extending window strut connected to a corresponding lower angled strut of a corresponding one of the two adjacent elongated cells, wherein the first and second axially extending window struts are connected together and to an upper axial strut, at their upper ends, via an upper edge of the commissure window that extends in the circumferential direction, and wherein the upper axial strut is connected at its upper end to an upper elongated strut junction forming a junction between upper, angled struts of the two adjacent elongated cells.

Example 13. The prosthetic heart valve of any example herein, particularly example 9 or example 10, wherein each commissure window is divided into a first open window portion and a second open window portion, each of the first and second open window portions configured to receive a different one of the commissure tabs of the commissure therein, wherein the first open window portion is formed between a central, axially extending strut and a first axial strut arm connected to and laterally offset from, in a circumferential direction relative to a circumference of the frame, a lower end of the axially extending strut, wherein the second open window portion is formed between the axially extending strut and a second axial strut arm connected to a laterally offset from the lower end of the axially extending strut, and wherein each of the first and second axial strut arms include an upper end that is free and configured to displace one or more of laterally and radially relative to the axially extending strut.

Example 14. The prosthetic heart valve of any example herein, particularly example 13, wherein the axially extending strut extends between a lower elongated strut junction of the frame and an upper elongated strut junction of the frame, the lower elongated strut junction being a junction between a first two angled struts of the two adjacent elongated cells, arranged at the outflow end of the frame, and the upper elongated strut junction being a junction between a second two angled struts of the two adjacent elongated cells, arranged at an opposite end of the two adjacent elongated cells from the first two angled struts.

Example 15. The prosthetic heart valve of any example herein, particularly any one of examples 9-14, wherein the annular frame is radially expandable to an expanded configuration.

Example 16. The prosthetic heart valve of any example herein, particularly example 15, wherein the expanded configuration has a diameter of 20 mm.

Example 17. The prosthetic heart valve of any example herein, particularly example 15, wherein the expanded configuration has a diameter in a range of 19-21 mm.

Example 18. A prosthetic heart valve, comprising: an annular frame including a plurality of interconnected struts defining a plurality of rows of cells, the plurality of rows of cells arranged between an outflow end and an inflow end of the frame and including an upper row of cells arranged at the outflow end that are elongated in an axial direction relative to cells of remaining rows of cells of the plurality of rows of cells, the axial direction relative to a central longitudinal axis of the frame; and a plurality of leaflets situated within the frame, each leaflet comprising opposing commissure tabs on opposite sides of the leaflet, each commissure tab being paired with an adjacent commissure tab of an adjacent leaflet to form a commissure; wherein struts defining the upper row of cells further define a plurality of open commissure windows that are open at an upper end and formed between adjacent elongated cells of the upper row of cells, each commissure window formed between two axially extending window struts that are spaced apart from one another in a circumferential direction, each commissure window comprising a lower region configured to receive commissure tabs of the commissure and an upper region arranged at the outflow end of the frame.

Example 19. The prosthetic heart valve of any example herein, particularly example 18, wherein each axially extending window strut of the two axially extending window struts defines an axial side of a different elongated cell of two adjacent elongated cells of the upper row of cells.

Example 20. The prosthetic heart valve of any example herein, particularly any one of examples 18-19, wherein each commissure window is closed at a lower end by a lateral window strut portion connecting lower ends of the two axially extending window struts together, the lower end arranged opposite the upper end in the axial direction.

Example 21. The prosthetic heart valve of any example herein, particularly example 20, wherein the lateral window strut portion further connects to angled struts defining a portion of a cell of a second row of cells arranged adjacent to the upper row of cells.

Example 22. The prosthetic heart valve of any example herein, particularly any one of examples 18-21, wherein each axially extending window strut of the two axially extending window struts of each commissure window includes a lower window strut portion and an upper window strut portion, wherein a first gap between upper window strut portions of the two axially extending window struts forms the upper region of the commissure window, and wherein a second gap between lower window strut portions of the two axially extending window struts forms the lower region of the commissure window.

Example 23. The prosthetic heart valve of any example herein, particularly example 22, wherein each upper window strut portion includes a recessed portion and wherein the first gap is greater than the second gap.

Example 24. The prosthetic heart valve of any example herein, particularly examples 22 and 23, further comprising a window fastener positioned within the upper region of each commissure window and configured to maintain a relatively constant distance between the two axially extending window struts.

Example 25. The prosthetic heart valve of any example herein, particularly example 24, wherein the window fastener comprises a middle portion and two opposing end portions, the middle portion extending between the two end portions, the two end portions having a width that is wider than a width of the middle portion, the widths arranged in the circumferential direction.

Example 26. The prosthetic heart valve of any example herein, particularly example 25, wherein the middle portion is arranged within the first gap, a first end portion of the two end portions extending circumferentially along and between radially outward facing sides of the upper window strut portions of the two axially extending window struts, and a second end portion of the two end portions extending circumferentially along and between radially inward facing sides of the upper window strut portions.

Example 27. The prosthetic heart valve of any example herein, particularly example 25, wherein lateral edges of the two end portions comprise a plurality of indentations adapted to receive a fastener therein.

Example 28. A prosthetic heart valve, comprising: an annular frame including a plurality of interconnected struts defining a plurality of rows of cells, the plurality of rows arranged between an outflow end and an inflow end of the frame and including an upper row of cells arranged at the outflow end that are elongated in an axial direction relative to cells of remaining rows of cells of the plurality of rows of cells, the axial direction relative to a central longitudinal axis of the frame; and a plurality of leaflets situated within the frame, each leaflet comprising opposing commissure tabs on opposite sides of the leaflet, each commissure tab being paired with an adjacent commissure tab of an adjacent leaflet to form a commissure; wherein struts defining the upper row of cells further define a plurality of open commissure windows that are open at a lower end and formed between adjacent elongated cells of the upper row of cells, within a lower portion of the adjacent elongated cells, each commissure window configured to receive a commissure therein and formed by two axially extending window struts that are spaced apart from one another in a circumferential direction, the two axially extending window struts connected to an upper axial strut via an upper edge of the commissure window and to lower angled struts of the upper row of cells, the upper axial strut and the two axially extending window struts together forming an axially extending strut defining an axial side of each of two adjacent elongated cells.

Example 29. The prosthetic heart valve of any example herein, particularly example 28, wherein the upper axial strut and the two axially extending window struts each extend in the axial direction, wherein the upper edge of the commissure window extends perpendicular to the upper axial strut, and wherein the circumferential direction is relative to a circumference of the frame.

Example 30. The prosthetic heart valve of any example herein, particularly any one of examples 28-29, wherein the upper edge is spaced away from the outflow end of the frame by the upper axial strut.

Example 31. The prosthetic heart valve of any example herein, particularly any one of examples 28-30, wherein the upper axial strut is arranged between an upper elongated strut junction and the upper edge, the upper elongated strut junction forming a junction between two angled struts arranged at the outflow end of the frame, each angled strut of the two angled struts at least partially forming an upper side of a respective one of the two adjacent elongated cells.

Example 32. The prosthetic heart valve of any example herein, particularly any one of examples 28-31, wherein each axially extending window strut of the two axially extending window struts includes a lower clamping portion, wherein the lower clamping portions of the two axially extending window struts are angled toward one another and define an opening therebetween, the opening being a lower opening of the commissure window.

Example 33. The prosthetic heart valve of any example herein, particularly example 32, wherein outer bends in the lower clamping portions of the two axially extending windows form a neck region that is configured to receive a fastener.

Example 34. A prosthetic heart valve, comprising: an annular frame including a plurality of interconnected struts defining a plurality of rows of cells, the plurality of rows of cells arranged between an outflow end and an inflow end of the frame and including an upper row of cells arranged at the outflow end that are elongated in an axial direction relative to cells of remaining rows of cells of the plurality of rows of cells, the axial direction relative to a central longitudinal axis of the frame; and a plurality of leaflets situated within the frame, each leaflet comprising opposing commissure tabs on opposite sides of the leaflet, each commissure tab being paired with an adjacent commissure tab of an adjacent leaflet to form a commissure; wherein struts defining the upper row of cells further define a plurality of open commissure windows, each commissure window being open at an upper end and formed between two adjacent elongated cells of the upper row of cells, within a lower portion of the two adjacent elongated cells, each commissure window split into a first open window portion and a second open window portion, each of the first and second open window portions configured to receive a different one of the commissure tabs of the commissure therein, wherein the first open window portion is formed between a central, axially extending strut and a first axial strut arm connected to and laterally offset, in a circumferential direction relative to a circumference of the frame, from the axially extending strut, and wherein the second open window portion is formed between the axially extending strut and a second axial strut arm connected to a laterally offset from the axially extending strut.

Example 35. The prosthetic heart valve of any example herein, particularly example 34, wherein the axially extending strut extends between a lower elongated strut junction of the frame and an upper elongated strut junction of the frame, the lower elongated strut junction being a junction between a first two angled struts of the two adjacent elongated cells, arranged at the outflow end of the frame, and the upper elongated strut junction being a junction between a second two angled struts of the two adjacent elongated cells, arranged at an opposite end of the two adjacent elongated cells from the first two angled struts.

Example 36. The prosthetic heart valve of any example herein, particularly example 35, wherein the first and second axial strut arms are arranged on opposite sides of the axially extending strut and are connected to the axially extending strut at a lower end of the axially extending strut that is arranged at the lower elongated strut junction.

Example 37. The prosthetic heart valve of any example herein, particularly example 35, wherein each of the first and second axial strut arms includes an axial portion extending parallel to the axially extending strut and a lateral portion that extends between the lower elongated strut junction and the axial portion.

Example 38. The prosthetic heart valve of any example herein, particularly example 37, wherein the lateral portion is arranged perpendicular to the axial portion.

Example 39. The prosthetic heart valve of any example herein, particularly examples 37 and 38, wherein each of the first and second axial strut arms further includes an upper clamping portion extending upward, toward the outflow end of the frame, from the axial portion and wherein the upper clamping portions of the first and second axial strut arms form a narrower, neck region at the open, upper end of the commissure window.

Example 40. The prosthetic heart valve of any example herein, particularly any one of examples 35-39, wherein each of the first and second axial strut arms includes a lower end directly connected to the axially extending strut at the lower elongated strut junction and an upper end that is free and is configured to displace laterally and/or radially relative to the axially extending strut.

Example 41. A prosthetic heart valve, comprising: an annular frame comprising: a plurality of interconnected struts defining a plurality of rows of cells, the plurality of rows of cells arranged between an outflow end and an inflow end of the frame and including an upper row of cells arranged at the outflow end and a lower row of cells arranged at the inflow end, wherein cells of the upper row of cells are elongated, in an axial direction relative to a central longitudinal axis of the frame, relative to cells of remaining rows of cells of the plurality of rows of cells, including the lower row of cells; and a plurality of commissure windows, each commissure window defined by a closed frame, the closed frame including a wider, first portion and a narrower, second portion, the first portion connected to an upper elongated strut junction of the frame via a bendable strut portion, the upper elongated strut junction being a junction between two angled struts of two adjacent elongated cells of the upper row of cells and an axially extending strut arranged between the two adjacent elongated cells, wherein the commissure window is configured to bend, via its bendable strut portion, from an extended configuration to a bent configuration where the commissure window overlaps the axially extending strut; and a leaflet assembly comprising a plurality of leaflets, each leaflet comprising opposing commissure tabs on opposite sides of the leaflet, each commissure tab being paired with an adjacent commissure tab of an adjacent leaflet to form a commissure, wherein each commissure is arranged within a respective second portion of a respective commissure window.

Example 42. The prosthetic heart valve of any example herein, particularly example 41, wherein the upper elongated strut junction is arranged at the outflow end of the frame and wherein when the commissure window is in the extended configuration, it extends axially outward and away from the outflow end of the frame.

Example 43. The prosthetic heart valve of any example herein, particularly any one of examples 41-42, wherein the bendable strut portion is configured to bend from the extended configuration to the bent configuration via manual force and wherein the bendable strut portion comprises a plastically deformable material.

Example 44. The prosthetic heart valve of any example herein, particularly any one of examples 41-43, wherein the bendable strut portion is axially aligned with the axially extending strut.

Example 45. The prosthetic heart valve of any example herein, particularly any one of examples 41-44, further comprising a wedge arranged within the first portion.

Example 46. The prosthetic heart valve of any example herein, particularly any one of examples 41-45, wherein the first portion is shorter than the second portion.

Example 47. A method for assembling a prosthetic heart valve comprising an annular frame and a leaflet assembly, comprising: bending a commissure window of the annular frame to a first bent orientation between a fully extended and a fully bent configuration, wherein the commissure window is defined by a closed strut frame including a wider, first portion and a narrower, second portion, the first portion connected to an upper elongated strut junction of the annular frame via a bendable strut portion, the upper elongated strut junction being a junction between two angled struts of two adjacent elongated cells of an upper row of elongated cells arranged at an outflow end of the annular frame and an axially extending strut arranged between the two adjacent elongated cells, wherein in the fully extended configuration the bendable strut portion is not bent and the commissure window extends in a axial direction, relative to a central longitudinal axis of the annular frame, outward and away from the outflow end of the annular frame, and wherein in the fully bent configuration the commissure window overlaps the axially extending strut; inserting a commissure, at least partially pre-assembled outside the annular frame, into the commissure window, wherein the commissure comprises a pair of secured together commissure tabs of adjacently arranged leaflets of the leaflet assembly; bending the commissure window to the fully bent configuration, so that the commissure window extends in parallel with the axially extending strut and is arranged radially inward of the axially extending strut; and securing the commissure window, in its fully bend configuration, to the annular frame.

Example 48. The method of any example herein, particularly example 47, wherein securing the commissure window to the annular frame includes securing a lateral edge of the second portion of the commissure window to the annular frame.

Example 49. The method of any example herein, particularly any one of examples 47-48, wherein the elongated cells of the upper row of elongated cells are elongated relative to cells of adjacent, remaining rows of cells of the annular frame, the remaining rows arranged further from the outflow end than the upper row of elongated cells.

Example 50. Example The method of any example herein, particularly any one of examples 47-49, wherein the inserting the commissure includes first inserting the commissure into the first portion of the strut frame of the commissure window and then sliding the commissure into the second portion of the strut frame of the commissure window.

Example 51. The method of any example herein, particularly any one of examples 47-50, further comprising inserting a wedge element into the first portion of the commissure window to further secure the commissure within the second portion of the commissure window.

Example 52. A method of assembling a prosthetic heart valve including an annular frame and a leaflet assembly, comprising: inserting a commissure, at least partially pre-assembled outside the annular frame, into a leaflet-receiving window of a commissure support element through an opening in the leaflet-receiving window, wherein the commissure comprises a pair of secured together commissure tabs of adjacently arranged leaflets of the leaflet assembly, wherein the leaflet-receiving window is formed by at least a portion of two axially extending members of the commissure support element that are open at a first end and connected at an opposing, second end by a connecting member, and wherein the commissure support element includes a coupling portion connected to the leaflet-receiving window and adapted to couple to a portion of the annular frame forming two adjacent cells in an upper row of cells of the annular frame, the upper row of cells arranged at an outflow end of the annular frame and having an elongated length, in an axial direction relative to a central longitudinal axis of the annular frame, relative to cells in remaining rows of cells of the annular frame; and attaching the commissure support element to the portion of the annular frame such that the leaflet-receiving window, and the commissure arranged therein, are arranged within a lower portion of the upper rows of cells and an open area that is unblocked from leaflets is formed within an upper portion of the upper row of cells.

Example 53. The method of any example herein, particularly example 52, wherein the attaching the commissure support element to the portion of the annular frame includes attaching the commissure support element to the two adjacent cells and arranging the leaflet-receiving window radially inward, relative to the central longitudinal axis, of an axial strut forming a common axial side of each of the two adjacent cells.

Example 54. The method of any example herein, particularly example 53, further comprising securing the connecting member of the commissure support element to a lower elongated strut junction of the upper row of cells via one or more fasteners, the lower elongated strut junction being a junction between two angled struts forming a lower edge of the two adjacent cells and a lower end of the axial strut.

Example 55. The method of any example herein, particularly example 53 or example 54, wherein the coupling portion of the commissure support element includes a pair of coupling members, each coupled to one of the two axially extending members by a different curved member of two curved members of the commissure support element.

Example 56. The method of any example herein, particularly example 55, wherein attaching the commissure support element to the portion of the annular frame includes hooking the two curved members around an upper edge of the two adjacent cells, the upper edge arranged at the outflow end of the frame and formed by two angled struts of the two adjacent cells.

Example 57. The method of any example herein, particularly example 56, further comprising securing the commissure support element to the annular frame via securing one or more fasteners around the pair of coupling members and upper axial portions of the two axially extending members of the commissure support element, wherein the upper axial portions extend from lower axial portions of the two axially extending members, the two lower axial portions forming the leaflet-receiving window.

Example 58. The method of any example herein, particularly example 55, wherein attaching the commissure support element to the portion of the annular frame includes hooking the two curved members through a central opening of a strut window of the axial strut and around a lower lateral strut portion of the strut window, wherein the strut window is a closed window formed by a plurality of connected strut portions, including the lower lateral strut portion, and is arranged along a length of the axial strut, a distance away from a junction between the angled struts of the two adjacent cells that form the upper edge and an upper end of the axial strut.

Example 59. The method of any example herein, particularly example 58, wherein attaching the commissure support element to the annular frame further comprises pinching the two curved members toward each other while inserting the pair of coupling members through the central opening of the strut window, and once the pair of coupling members are inserted through the central opening such that the two curved members are hooked around the lower lateral strut portion of the strut window, releasing the two curved members so that they are laterally biased against inner edges of axial strut portions of the strut window.

Example 60. A prosthetic heart valve, comprising: an annular frame including a plurality of interconnected struts defining a plurality of rows of cells, the plurality of rows of cells arranged between an outflow end and an inflow end of the frame and including an upper row of cells arranged at the outflow end that are elongated in an axial direction relative to cells of remaining rows of cells of the plurality of rows of cells, the axial direction relative to a central longitudinal axis of the frame; a plurality of leaflets situated within the frame, each leaflet comprising opposing commissure tabs on opposite sides of the leaflet, each commissure tab being paired with an adjacent commissure tab of an adjacent leaflet to form a commissure; and at least one commissure support element comprising a coupling portion and two axially extending members that are radially offset from the coupling portion and are laterally spaced apart from one another to form an open, leaflet-receiving window configured to receive the commissure, wherein the coupling portion is configured to couple to an upper edge of two adjacent cells of the upper row of cells, the upper edge arranged at the outflow end of the frame, and wherein the leaflet-receiving window is spaced apart, in the axial direction, from the upper edge.

Example 61. The prosthetic heart valve of any example herein, particularly example 60, wherein the upper edge of the two adjacent cells is formed by two angled struts of the two adjacent cells and wherein lower ends of the two angled struts are each connected to an axial strut forming a common axial side of each of the two adjacent cells, the axial strut arranged in parallel with the central longitudinal axis.

Example 62. The prosthetic heart valve of any example herein, particularly example 61, wherein the leaflet-receiving window is arranged radially inward of the axial strut when the commissure support element is coupled to the upper edge of the two adjacent cells of the upper row of cells.

Example 63. The prosthetic heart valve of any example herein, particularly example 62, wherein the axial strut divides the leaflet-receiving window into two open window portions, each of the two window portions defined between the axial strut and a different one of the two axially extending members, and wherein each of the two window portions is configured to receive one of the opposing commissure tabs of the commissure.

Example 64. The prosthetic heart valve of any example herein, particularly any one of examples 60-63, wherein the coupling portion includes a pair of coupling members, each coupled to one of the two axially extending members by a different curved member of two curved members of the commissure support element, and wherein when the coupling portion is coupled to the upper edge of the two adjacent cells, the pair of coupling members are arranged radially outward of the frame and the two axially extending members are arranged radially inward of the frame, relative to the central longitudinal axis.

Example 65. The prosthetic valve of any example herein, particularly any one of examples 60-64, wherein the leaflet-receiving window is further defined by a connecting member which extends between the two axially extending members, forming a closed end at a lower, first end of the leaflet-receiving window, and wherein an opposite, upper, second end of the leaflet-receiving window is open and configured to receive the commissure therethrough.

Example 66. The prosthetic heart valve of any example herein, particularly any one of examples 60-65, wherein the coupling portion includes a pair of coupling members, each coupled to one of the two axially extending members by a different curved member of two curved members of the commissure support element and wherein each curved member is configured to curve and hook around the upper edge of the two adjacent cells of the upper row of cells.

Example 67. The prosthetic heart valve of any example herein, particularly example 66, wherein each of the two axially extending members includes an upper axial portion and a lower axial portion, each lower axial portion extending between the connecting member and a bend between the lower axial portion and the upper axial portion and each upper axial portion extending between the bend and a corresponding curved member of the two curved members, wherein a first width between lower axial portions of the two axially extending members is wider than a second width between upper axial portions of the two axially extending members, and wherein the second width is a width of the leaflet-receiving window.

Example 68. The prosthetic heart valve of any example herein, particularly any one of examples 60-67, wherein the commissure support element is secured to the frame via one or more fasteners.

Example 69. A prosthetic heart valve, comprising: an annular frame including a plurality of interconnected struts defining a plurality of rows of cells, the plurality of rows arranged between an outflow end and an inflow end of the frame and including an upper row of cells arranged at the outflow end that are elongated in an axial direction relative to cells of remaining rows of cells of the plurality of rows of cells, the axial direction relative to a central longitudinal axis of the frame, wherein struts defining the upper row of cells include a plurality of axial struts, each axial strut forming a common, axial side of two adjacent cells of the upper row of cells, and wherein a portion of the plurality of axial struts are window axial struts, each window axial strut including a closed strut window positioned a distance away from an upper end of the window axial strut, along a length of the window axial strut; a plurality of leaflets situated within the frame, each leaflet comprising opposing commissure tabs on opposite sides of the leaflet, each commissure tab being paired with an adjacent commissure tab of an adjacent leaflet to form a commissure; and at least one commissure support element comprising a coupling portion and two axially extending members that are radially offset from the coupling portion and are laterally spaced apart from one another to form an open, leaflet-receiving window configured to receive the commissure, wherein the coupling portion is configured to couple to the strut window.

Example 70. The prosthetic heart valve of any example herein, particularly example 69, wherein when the commissure support element is coupled to the strut window of the window axial strut, the leaflet-receiving window is positioned directly, radially inward of a lower strut portion of the window axial strut, the strut window arranged between the lower strut portion and an upper strut portion of the window axial strut, the lower strut portion connected to angled struts forming a lower edge of the two adjacent cells of the upper row of cells and the upper strut portion connected to angled struts forming an upper edge of the two adjacent cells.

Example 71. The prosthetic heart valve of any example herein, particularly example 70, wherein a length of the upper strut portion is shorter than a length of the lower strut portion and wherein the length of the lower strut portion corresponds to a length of the leaflet-receiving window of the commissure support element.

Example 72. The prosthetic heart valve of any example herein, particularly any one of examples 69-71, wherein the distance at which the closed strut window is spaced away from the upper end of the window axial strut is selected so that, when the commissure support element is coupled to the strut window, the leaflet-receiving window is positioned closer to a lower end of the two adjacent cells than an upper end of the two adjacent cells, the upper end arranged at the outflow end of the annular frame.

Example 73. The prosthetic heart valve of any example herein, particularly any one of examples 69-72, wherein the coupling portion of the commissure support element includes a pair of coupling members that are radially offset from the two axially extending members, the pair of coupling members including a first coupling member coupled to a first axial member of the two axially extending members by a first curved member and a second coupling member coupled to a second axial member of the two axially extending members by a second curved member.

Example 74. The prosthetic heart valve of any example herein, particularly example 73, wherein the strut window includes a plurality of connected strut portions that define a central opening of the strut window, a width of the central opening sized to accept the first and second curved members.

Example 75. The prosthetic heart valve of any example herein, particularly example 74, wherein the width of the central opening is the same or smaller than a free-state distance between outer edges of the first and second curved members.

Example 76. A prosthetic heart valve, comprising: an annular frame, comprising: a plurality of interconnected struts defining a plurality of rows of cells, the plurality of rows arranged between an outflow end and an inflow end of the frame and including an upper row of cells arranged at the outflow end and a lower row of cells arranged at the inflow end, wherein cells of the upper row of cells are elongated, in an axial direction relative to a central longitudinal axis of the frame, relative to cells of remaining rows of cells of the plurality of rows of cells, including the lower row of cells; and a plurality of commissure windows formed between axially extending window struts of the frame, each commissure window arranged between axially extending window struts of two adjacent elongated cells of the upper row of open cells, each commissure window having a commissure-receiving portion that is spaced away from an upper end of the two adjacent elongated cells, the upper end arranged at the outflow end of the frame; and a leaflet assembly comprising a plurality of leaflets, each leaflet comprising opposing commissure tabs on opposite sides of the leaflet, each commissure tab being paired with an adjacent commissure tab of an adjacent leaflet to form a commissure, wherein each commissure is arranged within the commissure-receiving portion of a respective commissure window, and wherein when each commissure is arranged within the commissure-receiving portion of the respective commissure window the commissure is arranged within a lower portion of the upper rows of cells and an open area that is unblocked from leaflets is formed within an upper portion of the upper row of cells, the upper portion arranged closer to the outflow end than the lower portion.

Example 77. A prosthetic heart valve, comprising: an annular frame, comprising: a plurality of interconnected struts defining a plurality of rows of cells, the plurality of rows arranged between an outflow end and an inflow end of the frame and including an upper row of cells arranged at the outflow end and a lower row of cells arranged at the inflow end; and a plurality of commissure windows formed between axially extending window struts of the frame, each commissure window arranged between axially extending window struts of two adjacent cells of the upper row of cells, each commissure window having a commissure-receiving portion that is spaced away from an upper end of the two adjacent cells, the upper end arranged at the outflow end of the frame, each commissure window having an open end; and a leaflet assembly comprising a plurality of leaflets, each leaflet comprising opposing commissure tabs on opposite sides of the leaflet, each commissure tab being paired with an adjacent commissure tab of an adjacent leaflet to form a commissure, wherein each commissure is arranged within the commissure-receiving portion of a respective commissure window, and wherein the open end of the commissure window is configured to receive the commissure therethrough.

Example 78. The prosthetic heart valve of any example herein, particularly example 77, wherein the open end of each commissure window is formed by upper or lower ends of the axially extending window struts that are spaced apart from one another, in a circumferential direction, and not directly connected to one another, the circumferential direction relative to a circumference of the frame and arranged tangent to the axial direction.

Example 79. The prosthetic heart valve of any example herein, particularly examples 77 or 78, wherein each commissure window is formed between a first axially extending window strut of a first cell of the two adjacent cells and a second axially extending window strut of a second cell of the two adjacent cells, the first and second axially extending window struts spaced apart from one another at their upper ends and along their lengths and connected together at their lower ends by a lateral window strut portion, wherein each of the first and second axially extending window struts comprise a lower window strut portion and an upper window strut portion, and wherein the commissure-receiving portion of the commissure is formed in a space between the lower window strut portion of the first axially extending window strut and the lower window strut portion of the second axially extending window strut.

Example 80. The prosthetic heart valve of any example herein, particularly examples 77 or 78, wherein each commissure window is formed between a first axially extending window strut and a second axially extending window strut that are spaced apart from one another in the circumferential direction at their lower ends and along their length, the lower end of each of the first and second axially extending window strut connected to a corresponding lower angled strut of a corresponding one of the two adjacent cells, wherein the first and second axially extending window struts are connected together and to an upper axial strut, at their upper ends, via an upper edge of the commissure window that extends in the circumferential direction, and wherein the upper axial strut is connected at its upper end to an upper strut junction forming a junction between upper, angled struts of the two adjacent cells.

Example 81. The prosthetic heart valve of any example herein, particularly examples 77 or 78, wherein each commissure window is divided into a first open window portion and a second open window portion, each of the first and second open window portions configured to receive a different one of the commissure tabs of the commissure therein, wherein the first open window portion is formed between a central, axially extending strut and a first axial strut arm connected to and laterally offset from, in a circumferential direction relative to a circumference of the frame, a lower end of the axially extending strut, wherein the second open window portion is formed between the axially extending strut and a second axial strut arm connected to a laterally offset from the lower end of the axially extending strut, and wherein each of the first and second axial strut arms include an upper end that is free and configured to displace one or more of laterally and radially relative to the axially extending strut.

Example 82. The prosthetic heart valve of any example herein, particularly example 81, wherein the axially extending strut extends between a lower strut junction of the frame and an upper strut junction of the frame, the lower strut junction being a junction between a first two angled struts of the two adjacent cells, arranged at the outflow end of the frame, and the upper strut junction being a junction between a second two angled struts of the two adjacent cells, arranged at an opposite end of the two adjacent cells from the first two angled struts.

Example 83. The prosthetic heart valve of any example herein, particularly any one of example 77-82, wherein the annular frame is radially expandable to an expanded configuration.

Example 84. The prosthetic heart valve of any example herein, particularly any one of examples 77-83, wherein the cells of the upper row of cells are elongated in an axial direction, relative to cells of the lower row of cells.

Example 85. A prosthetic heart valve, comprising: an annular frame including a plurality of interconnected struts defining a plurality of rows of cells, the plurality of rows of cells arranged between an outflow end and an inflow end of the frame and including an upper row of cells arranged at the outflow end; and a plurality of leaflets situated within the frame, each leaflet comprising opposing commissure tabs on opposite sides of the leaflet, each commissure tab being paired with an adjacent commissure tab of an adjacent leaflet to form a commissure; wherein struts defining the upper row of cells further define a plurality of open commissure windows that are open at an upper end and formed between adjacent cells of the upper row of cells, each commissure window formed between two axially extending window struts that are spaced apart from one another in a circumferential direction, each commissure window comprising a lower region configured to receive commissure tabs of the commissure.

Example 86. The prosthetic heart valve of any example herein, particularly example 85, wherein each axially extending window strut of the two axially extending window struts defines an axial side of a different cell of two adjacent cells of the upper row of cells.

Example 87. The prosthetic heart valve of any example herein, particularly examples 85 or 86, wherein each commissure window is closed at a lower end by a lateral window strut portion connecting lower ends of the two axially extending window struts together, the lower end arranged opposite the upper end in the axial direction.

Example 88. The prosthetic heart valve of any example herein, particularly example 87, wherein the lateral window strut portion further connects to angled struts defining a portion of a cell of a second row of cells arranged adjacent to the upper row of cells.

Example 89. The prosthetic heart valve of any example herein, particularly any one of examples 85-88, wherein each axially extending window strut of the two axially extending window struts of each commissure window includes a lower window strut portion and an upper window strut portion, wherein a first gap between upper window strut portions of the two axially extending window struts forms the upper region of the commissure window, wherein a second gap between lower window strut portions of the two axially extending window struts forms the lower region of the commissure window, wherein each upper window strut portion includes a recessed portion, and wherein the first gap is greater than the second gap.

Example 90. The prosthetic heart valve of any example herein, particularly example 89, further comprising a window fastener positioned within the upper region of each commissure window and configured to maintain a relatively constant distance between the two axially extending window struts.

Example 91. The prosthetic heart valve of any example herein, particularly example 90, wherein the window fastener comprises a middle portion and two opposing end portions, the middle portion extending between the two end portions, the two end portions having a width that is wider than a width of the middle portion, the widths arranged in the circumferential direction, and wherein the middle portion is arranged within the first gap, a first end portion of the two end portions extending circumferentially along and between radially outward facing sides of the upper window strut portions of the two axially extending window struts, and a second end portion of the two end portions extending circumferentially along and between radially inward facing sides of the upper window strut portions.

Example 92. A prosthetic heart valve, comprising: an annular frame including a plurality of interconnected struts defining a plurality of rows of cells, the plurality of rows arranged between an outflow end and an inflow end of the frame and including an upper row of cells arranged at the outflow end; and a plurality of leaflets situated within the frame, each leaflet comprising opposing commissure tabs on opposite sides of the leaflet, each commissure tab being paired with an adjacent commissure tab of an adjacent leaflet to form a commissure; wherein struts defining the upper row of cells further define a plurality of open commissure windows formed between adjacent cells of the upper row of cells, each commissure window configured to receive a commissure therein and formed by two axially extending window struts that are spaced apart from one another in a circumferential direction, wherein the commissure windows are offset axially toward an upstream/inflow end of the upper row of cells.

Example 93. The prosthetic heart valve of any example herein, particularly example 92, wherein the two axially extending window struts are connected to an upper axial strut via an upper edge of the commissure window and to lower angled struts of the upper row of cells, the upper axial strut and the two axially extending window struts together forming an axially extending frame portion defining an axial side of each of two adjacent cells.

Example 94. The prosthetic heart valve of any example herein, particularly example 93, wherein the upper axial strut and the two axially extending window struts each extend in the axial direction, wherein the upper edge of the commissure window extends perpendicular to the upper axial strut, and wherein the circumferential direction is relative to a circumference of the frame.

Example 95. The prosthetic heart valve of any example herein, particularly examples 93 or 94, wherein the upper edge is spaced away from the outflow end of the frame by the upper axial strut.

Example 96. The prosthetic heart valve of any example herein, particularly example 95, wherein a length of the upper axial strut is selected such that an axial distance between the outflow end of the frame and outflow edges of the plurality of leaflets is in a range of 2-3 mm.

Example 97. The prosthetic heart valve of any example herein, particularly any one of examples 93-96, wherein the upper axial strut is arranged between an upper strut junction and the upper edge, the upper strut junction forming a junction between two angled struts arranged at the outflow end of the frame, each angled strut of the two angled struts at least partially forming an upper side of a respective one of the two adjacent cells.

Example 98. The prosthetic heart valve of any example herein, particularly any one of examples 93-97, wherein each commissure window is open at a lower end and wherein each axially extending window strut of the two axially extending window struts includes a lower clamping portion, wherein the lower clamping portions of the two axially extending window struts are angled toward one another and define an opening therebetween, the opening being a lower opening of the commissure window.

Example 99. The prosthetic heart valve of any example herein, particularly example 98, wherein outer bends in the lower clamping portions of the two axially extending windows form a neck region that is configured to receive a fastener.

In view of the many possible embodiments to which the principles of the disclosed technology may be applied, it should be recognized that the illustrated embodiments are only preferred examples of the disclosed technology and should not be taken as limiting the scope of the claimed subject matter. Rather, the scope of the claimed subject matter is defined by the following claims and their equivalents.

We claim:

1. A prosthetic heart valve, comprising:
an annular frame, comprising:
a plurality of interconnected struts defining a plurality of rows of cells, the plurality of rows arranged between an outflow end and an inflow end of the frame and including an upper row of cells arranged at the outflow end; and
a plurality of commissure windows formed between axially extending window struts of the frame, each commissure window arranged between axially extending window struts of two adjacent cells of the upper row of cells, each commissure window having a commissure-receiving portion that is spaced away from an upper end of the two adjacent cells, the upper end arranged at the outflow end of the frame, each commissure window having an open end; and
a leaflet assembly comprising a plurality of leaflets, each leaflet comprising opposing commissure tabs on opposite sides of the leaflet, each commissure tab being paired with an adjacent commissure tab of an adjacent leaflet to form a commissure, wherein each commissure is arranged within the commissure-receiving portion of a respective commissure window, and wherein the open end of the commissure window is configured to receive the commissure therethrough.

2. The prosthetic heart valve of claim 1, wherein the open end of each commissure window is formed by upper or lower ends of the axially extending window struts that are spaced apart from one another, in a circumferential direction, and not directly connected to one another, the circumferential direction relative to a circumference of the frame and arranged tangent to the axial direction.

3. The prosthetic heart valve of claim 1, wherein each commissure window is formed between a first axially extending window strut of a first cell of the two adjacent cells and a second axially extending window strut of a second cell of the two adjacent cells, the first and second axially extending window struts spaced apart from one another at their upper ends and along their lengths and connected together at their lower ends by a lateral window strut portion, wherein each of the first and second axially extending window struts comprise a lower window strut portion and an upper window strut portion, and wherein the commissure-receiving portion of the commissure is formed in a space between the lower window strut portion of the first axially extending window strut and the lower window strut portion of the second axially extending window strut.

4. The prosthetic heart valve of claim 1, wherein each commissure window is formed between a first axially extending window strut and a second axially extending window strut that are spaced apart from one another in the circumferential direction at their lower ends and along their length, the lower end of each of the first and second axially extending window strut connected to a corresponding lower angled strut of a corresponding one of the two adjacent cells, wherein the first and second axially extending window struts are connected together and to an upper axial strut, at their upper ends, via an upper edge of the commissure window that extends in the circumferential direction, and wherein the upper axial strut is connected at its upper end to an upper strut junction forming a junction between upper, angled struts of the two adjacent cells.

5. The prosthetic heart valve of claim 1, wherein each commissure window is divided into a first open window portion and a second open window portion, each of the first and second open window portions configured to receive a different one of the commissure tabs of the commissure therein, wherein the first open window portion is formed between a central, axially extending strut and a first axial strut arm connected to and laterally offset from, in a circumferential direction relative to a circumference of the frame, a lower end of the axially extending strut, wherein the second open window portion is formed between the axially extending strut and a second axial strut arm connected to a laterally offset from the lower end of the axially extending strut, and wherein each of the first and second axial strut arms include an upper end that is free and configured to displace one or more of laterally and radially relative to the axially extending strut.

6. The prosthetic heart valve of claim 5, wherein the axially extending strut extends between a lower strut junction of the frame and an upper strut junction of the frame, the lower strut junction being a junction between a first two angled struts of the two adjacent cells, arranged at the outflow end of the frame, and the upper strut junction being a junction between a second two angled struts of the two adjacent cells, arranged at an opposite end of the two adjacent cells from the first two angled struts.

7. The prosthetic heart valve of claim 1, wherein the annular frame is radially expandable to an expanded configuration.

8. A prosthetic heart valve, comprising:
an annular frame including a plurality of interconnected struts defining a plurality of rows of cells, the plurality of rows of cells arranged between an outflow end and an inflow end of the frame and including an upper row of cells arranged at the outflow end; and
a plurality of leaflets situated within the frame, each leaflet comprising opposing commissure tabs on opposite sides of the leaflet, each commissure tab being paired with an adjacent commissure tab of an adjacent leaflet to form a commissure;
wherein struts defining the upper row of cells further define a plurality of open commissure windows that are open at an upper end and formed between adjacent cells of the upper row of cells, each commissure window formed between two axially extending window struts that are spaced apart from one another in a circumferential direction, each commissure window comprising a lower region configured to receive commissure tabs of the commissure.

9. The prosthetic heart valve of claim 8, wherein each axially extending window strut of the two axially extending window struts defines an axial side of a different cell of two adjacent cells of the upper row of cells.

10. The prosthetic heart valve of claim 8, wherein each commissure window is closed at a lower end by a lateral window strut portion connecting lower ends of the two axially extending window struts together, the lower end arranged opposite the upper end in the axial direction.

11. The prosthetic heart valve of claim 10, wherein the lateral window strut portion further connects to angled struts defining a portion of a cell of a second row of cells arranged adjacent to the upper row of cells.

12. The prosthetic heart valve of claim 8, wherein each axially extending window strut of the two axially extending window struts of each commissure window includes a lower window strut portion and an upper window strut portion, wherein a first gap between upper window strut portions of the two axially extending window struts forms the upper region of the commissure window, wherein a second gap between lower window strut portions of the two axially extending window struts forms the lower region of the commissure window, wherein each upper window strut portion includes a recessed portion, and wherein the first gap is greater than the second gap.

13. The prosthetic heart valve of claim 12, further comprising a window fastener positioned within the upper region of each commissure window and configured to maintain a relatively constant distance between the two axially extending window struts.

14. The prosthetic heart valve of claim 13, wherein the window fastener comprises a middle portion and two opposing end portions, the middle portion extending between the two end portions, the two end portions having a width that is wider than a width of the middle portion, the widths arranged in the circumferential direction, and wherein the middle portion is arranged within the first gap, a first end portion of the two end portions extending circumferentially along and between radially outward facing sides of the upper window strut portions of the two axially extending window struts, and a second end portion of the two end portions extending circumferentially along and between radially inward facing sides of the upper window strut portions.

15. A prosthetic heart valve, comprising:
an annular frame including a plurality of interconnected struts defining a plurality of rows of cells, the plurality of rows arranged between an outflow end and an inflow end of the frame and including an upper row of cells arranged at the outflow end; and
a plurality of leaflets situated within the frame, each leaflet comprising opposing commissure tabs on opposite sides of the leaflet, each commissure tab being paired with an adjacent commissure tab of an adjacent leaflet to form a commissure;
wherein struts defining the upper row of cells further define a plurality of open commissure windows formed between adjacent cells of the upper row of cells, each commissure window configured to receive a commissure therein and formed by two axially extending window struts that are spaced apart from one another in a circumferential direction, wherein the commissure windows are offset axially toward an inflow end of the upper row of cells.

16. The prosthetic heart valve of claim 15, wherein the two axially extending window struts are connected to an upper axial strut via an upper edge of the commissure window and to lower angled struts of the upper row of cells, the upper axial strut and the two axially extending window struts together forming an axially extending frame portion defining an axial side of each of two adjacent cells.

17. The prosthetic heart valve of claim 16, wherein the upper axial strut and the two axially extending window struts each extend in the axial direction, wherein the upper edge of the commissure window extends perpendicular to the upper axial strut, and wherein the circumferential direction is relative to a circumference of the frame.

18. The prosthetic heart valve of claim 16, wherein the upper edge is spaced away from the outflow end of the frame by the upper axial strut.

19. The prosthetic heart valve of claim 18, wherein a length of the upper axial strut is selected such that an axial distance between the outflow end of the frame and outflow edges of the plurality of leaflets is in a range of 2-3 mm.

20. The prosthetic heart valve of claim 16, wherein the upper axial strut is arranged between an upper strut junction and the upper edge, the upper strut junction forming a junction between two angled struts arranged at the outflow end of the frame, each angled strut of the two angled struts at least partially forming an upper side of a respective one of the two adjacent cells.

21. The prosthetic heart valve of claim 16, wherein each commissure window is open at a lower end and wherein each axially extending window strut of the two axially extending window struts includes a lower clamping portion, wherein the lower clamping portions of the two axially extending window struts are angled toward one another and define an opening therebetween, the opening being a lower opening of the commissure window.

22. The prosthetic heart valve of claim 21, wherein outer bends in the lower clamping portions of the two axially extending windows form a neck region that is configured to receive a fastener.
